(19) United States
(12) Patent Application Publication
(10) Pub. No.: US 2018/0230507 A1
(43) Pub. Date: Aug. 16, 2018

(54) HIGH-THROUGHPUT METHODS FOR OBTAINING SEED TREATMENT-TOLERANT MICROORGANISMS

(71) Applicant: Monsanto Technology LLC, St. Louis, MO (US)

(72) Inventors: Janne S. Kerovuo, San Diego, CA (US); Ryan T. McCann, San Diego, CA (US)

(21) Appl. No.: 15/753,130

(22) PCT Filed: Aug. 18, 2016
(86) PCT No.: PCT/US16/47486

§ 371 Date: Feb. 15, 2018

Related U.S. Application Data
(60) Provisional application No. 62/207,868, filed on Aug. 20, 2015.

(51) Int. Cl. C12Q 1/02; A01N 63/00; C12Q 1/24; C12R 1/01; C05F 11/08; C05G 3/02

(52) U.S. Cl. CPC C12Q 1/025; A01N 63/00; C05G 3/02; C12R 1/01; C05F 11/08; C12Q 1/24

(57) ABSTRACT

Provided herein are methods for identifying and obtaining environmental isolates of microorganisms that maintain viability following seed treatment components or seed treatment processes and storage.

### CROSS-REFERENCE TO RELATED APPLICATIONS

This application claims the benefit of U.S. Provisional Application No. 62/207,868, filed Aug. 20, 2015, herein incorporated by reference in its entirety.

### BACKGROUND OF THE INVENTION

### Field of the Invention

Provided herein are high-throughput methods of obtaining microorganisms capable of remaining viable following seed treatment processes.

### Description of Related Art

Agricultural crop production often utilizes chemical treatments of seeds. Often, such treatments are relied upon to impart disease or pest resistance properties to the seed or resulting plant. Application of seed treatments before planting may reduce damage to the seed during storage, germination, or planting, and also protect the emerging plant. This can help achieve uniform stand establishment, which not only has the benefit of protecting an investment in seeds themselves, but also maximizes plant performance per unit land. Increasingly, microbes that are beneficial to seeds and plants are included in seed treatments and may impart a wide range of beneficial agronomic traits. Microorganisms may find use, for example as plant inoculants, soil amendments, biocontrol agents or plant growth regulators

Despite advances in the field, there remains a need to identify additional microorganisms for use in plant seed treatments.

### BRIEF SUMMARY OF THE INVENTION

In one aspect, provided herein is a high-throughput method for obtaining a microorganism comprising the steps of: a) obtaining a plurality of microorganisms associated with a crop plant in a growth environment; b) applying a plurality of microorganisms to a seed or surrogate thereof; c) storing the seed, or surrogate thereof under conditions wherein one or more members of the plurality of microorganisms becomes inviable; d) placing the seed, or surrogate thereof, in a solution; and e) identifying from the solution at least a first microorganism remaining viable following step (c). In one embodiment, step a) comprises generating a microbial cell suspension from a crop plant and/or the growth environment. Alternatively, the method may comprise concentrating the microbial cell suspension prior to the step b). In specific embodiments, step e) comprises plating the solution onto a growth medium and selecting a colony comprising the microorganism.

In a particular aspect of the invention, obtaining a plurality of microorganisms associated with a crop plant in a growth environment comprises i) generating a microbial cell suspension from the crop plant and/or the growth environment; ii) plating the microbial cell suspension onto a growth medium; iii) selecting microbial colonies; and iv) producing the plurality of microorganisms by combining members of the selected microbial colonies in step iii). In a method of the invention, a growth environment may comprise soil from an agricultural field, or may comprise a non-agriculture environment. In particular embodiments, a plurality of microorganisms used with the invention are from a crop plant rhizosphere, endosphere, phyllosphere, or any combination thereof. The plurality of microorganisms may also be obtained from tissue of a crop plant.

In a specific embodiment, a method of the invention further comprises the step of identifying at least a first beneficial trait that the microorganism is capable of conferring upon plants of a crop plant species. The crop plant species may or may not be of the same species as a seed used in a method of the invention. The crop plant species may be a dicotyledonous plant species, including, but not limited to, alfalfa, beans, beet, broccoli, cabbage, carrot, cauliflower, celery, Chinese cabbage, cotton, cucumber, eggplant, flax, lettuce, lupine, melon, pea, pepper, peanut, potato, pumpkin, radish, rapeseed, spinach, soybean, squash, sugarbeet, sunflower, tomato, and watermelon. The crop plant species may also be a monocotyledonous plant species, including but not limited to barley, corn, leek, onion, rice, sorghum, sweet corn, wheat, rye, millet, sugarcane, oat, triticale, switchgrass, and turfgrass.

In further embodiments of a method of the invention, a microorganism remaining viable is a gram-negative, non-spore forming bacterium or a gram-positive, spore forming or non-spore forming bacterium. In other embodiments, storing of a seed, or surrogate thereof, is carried out for from about 1 hour to about 1 year, is carried out at ambient temperature, or is carried out at above or below ambient temperature.

In still further embodiments of the invention, a plurality of microorganisms are applied to a seed or surrogate thereof and a seed treatment is applied to the seed or surrogate thereof prior to, concurrently with, or after applying the plurality of microorganisms. The seed treatment may or may not comprise the plurality of microorganisms. In one embodiment, the seed treatment is applied to the seed or surrogate thereof prior to or after applying the plurality of microorganisms. In another embodiment, the seed treatment comprises a fungicide, biocide, insecticide, herbicide, miticide, rodenticide, nematicides, plant growth regulators, and micronutrients, or a combination thereof. In yet another embodiment, the seed treatment comprises a polymer, colorant, binder, adhesive, adherent, dispersant, surfactant, nutrient, coating agent, wetting agent, buffering agent, polysaccharide, and filler, or a combination thereof.

### BRIEF DESCRIPTION OF THE DRAWINGS

FIG. 1. Schematic of high-throughput Method B1 and Method B2 for discovery and isolation of seed treatment-tolerant microorganisms.

FIG. 2A. Representative histogram of diversity discovered via Method B1 by taxonomic order.

FIG. 2B. Representative histogram of diversity discovered via Method B1 by taxonomic class.

FIG. 2C. Representative histogram of diversity discovered via Method B1 by number of genera per taxonomic class.

FIG. 3A. Representative histogram of diversity discovered via Method B2 by taxonomic order.

FIG. 3B. Representative histogram of diversity discovered via Method B2 by taxonomic class.

FIG. 3C. Representative histogram of diversity discovered via Method B2 by number of genera per taxonomic class.

FIG. 4A. Representative histogram of diversity discovered via Method Bland Method B2 by taxonomic order.

FIG. 4B. Representative histogram of diversity discovered via Method B land Method B2by taxonomic class.

FIG. 4C. Representative histogram of diversity discovered via Method B land Method B2 by number of genera per taxonomic class.

### DETAILED DESCRIPTION OF THE INVENTION

Provided herein are methods for obtaining microorganisms that are able to both colonize plants of interest, and survive the conditions associated with seed treatment components and seed treatment processes. The inventors have found, for example, that the viability of microbes incorporated into seed treatments can vary between different species of microbes and potentially between different strains of the same species. Which microorganisms or strains will tolerate a seed treatment component or seed treatment process has therefore generally been unpredictable to date. Moreover, a lag time of at least one to several months, to even years can exist between the treating of seeds and their eventual planting. This has significantly impacted the ability to identify microorganisms that can benefit agriculture due to a loss of viability of some strains prior to planting. This is compounded by difficulties in predicting which strains will exhibit a loss of viability ahead of time, and also the need to introduce improved beneficial microorganisms each growing season. Testing each candidate microbe individually is time-consuming and impractical.

In previous studies, the present inventors individually tested hundreds of gram negative bacterial strains associated with crop plants for seed treatment component tolerance and results indicated that the majority of species typically isolated from plant-microbe communities will not survive to the point of detection after 1-2 weeks when applied to seeds at 10⁶ to 10⁷ colony forming units (CFUs) per seed. Furthermore, results revealed that some of the most abundant microbes in the rhizosphere experience a near-total loss of viability following seed treatment, regardless of the concentration of the microbe applied to the seed. The methods provided herein overcome such deficiencies, permitting the identification of large populations of microorganisms in a short period of time and providing large numbers of microbes that are capable of surviving seed treatment components and processes that can be further tested for the microbes' beneficial effects on agricultural crop plants.

It was surprisingly found that the methods disclosed herein permit identification of not just hardy and fast-growing microorganisms, such as spore-forming bacteria, but also a diverse collection of other microorganisms, such as non-spore forming bacteria. In a non-limiting example, a plurality of Gram-negative bacteria, which often do not survive the seed treatment process or components, or are difficult to test for the ability to persist, are isolated from the rhizosphere of crop plants and screened in accordance with the disclosed methods.

In one embodiment, a high-throughput method of isolating, selecting, and identifying seed treatment-tolerant microorganisms is provided capable of beneficial use in agriculture even after storage within a seed coating. In specific embodiments, the method comprises obtaining a plurality of microorganisms endemic to a selected environment or environments and identifying those capable of surviving seed treatment components and seed treatment processes and reagents. In one embodiment, a pool of microbes is applied to seeds at a pre-determined rate. In other embodiments, a concentrated microbial cell suspension is produced containing a diverse pool of microbes to be applied directly to seeds. The suspension can be a representative extract of all of the bacteria, fungi, and archaea present in a microbiota, including, but not limited to, those found in soils, plant tissues, and bodies of water. The cell suspension can also be derived from an artificially assembled pool of microbes that were cultured previously. These cell suspensions can be used to inoculate seeds in order to identify those capable of surviving the seed treatment process.

In additional embodiments, polymers, colorants, pesticides, including fungicides, biocides, insecticides, herbicide, miticides, rodenticides, nematicides, or other components that may be used in seed treatment preparations are applied to the seeds in a similar fashion to select for microorganisms capable of surviving various seed treatment components and processes. In certain embodiments, strains of microbes that survive seed treatment and incubation are cultured and archived for future use in field trial screens for beneficial effects on plant growth.

Suitable methods and compositions are known in the art for producing and applying seed treatments with specific ingredients, such as one or more of a pesticide, including for example, nematicide, insecticide, fungicide, inoculant, or formulation component, including for example, a coating agent or protectant, so as to provide protection against seedling and seed diseases, early-season insects and pests and the like. Pre-treatment of seeds prior to storage and planting can maximize early-season plant stand, uniformity, and vigor for higher yield potential, for example.

Usage rates for seed treatment components used in the present invention may be those standard in the art for the particular geographic region where a seed is to be planted or for the intended benefit intended to be achieved by the treatment. Standard ranges are well known in the art and are often guided by local regulatory requirements, which may set minimum and maximum levels for applications to commercial seed. An example of a suitable range for a particular pesticide product in the seed treatment tolerance methods described herein is the range given on the product label for use in commercial applications. Alternatively, the high-throughput methods described herein may use an increased amount of any one or more seed treatment component that could potentially impact the viability of a microorganism to provide a more stringent or compact test for the ability to maintain viability following seed treatment.

The amount of coating agent, microbial, or protectant may likewise depend on various factors, such as the compounds employed, the seed type treated, the proposed planting conditions, and the expected climactic conditions. Using the guidance provided herein a skilled person will be able to determine the specific amounts which would be suitable for use according to the invention. Often, formulated products (as opposed to pure active ingredient) are used in seed treatments. For convenience of supply and ease of use, formulated products may be advantageously used in the methods described herein.

In some embodiments, a vast array of microbes—representing countless microbial species and variants—contained in plant/soil extracts are isolated, selected, and optionally identified, and tested for seed treatment tolerance in a single assay. In another aspect of the invention, seeds are directly coated with extracts taken from plants and soils of interest to avoid a media bias present when performing a microbial isolation step. In particular embodiments, a large number of different crops, grown in a variety of soils, are harvested so that the associated microbes can be applied to seeds of a given species or variety thereof and microbes that colonize these plants of interest, and survive the process of being dosed onto the seed, are thus selected.

The methods of the instant invention permit, for the first time, preferential enrichment of strains from environmental samples that colonize plants of interest, and that are also capable of surviving incorporation into seed treatments. Established isolation protocols are generally directed towards discovering microorganisms that exhibit specific growth patterns, utilize certain substrates, or test positive for desired biochemical activities. The described methods allow for the user to vary the incubation time that microbes spend in seed treatment compositions, which gives greater control over the degree of stability required and is more applicable to real world scenarios. Where desired, the starting microbes analyzed in accordance with the invention can be enriched towards species that colonize a particular crop plant of interest or variety thereof.

The insertion of a seed treatment step into the isolation procedure provides the advantage of not having to individually test each microbial candidate for seed treatment tolerance prior to field testing, which is the current paradigm for determining whether specific microbes are commercially viable. The result is an increased efficiency over traditional methods. The methods disclosed herein offer a rapid, streamlined process for identifying such microbes, avoiding the need for lengthy or individualized efforts and saving significant resources.

### Definitions

Unless otherwise noted, terms are to be understood according to conventional usage by those of ordinary skill in the relevant art.

In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a,” “an,” and “the” include plural references unless the context clearly dictates otherwise. For example, the term “a cell” includes one or more cells, including mixtures thereof.

As used herein, the term “high throughput” refers to a process in which numerous microbes, including any microbial species and/or variants from any microbial community sample or from multiple different samples (e.g., environmental samples from the rhizosphere, endosphere, or soil), are tested in single protocol, for example an array of microbes representing numerous microbial species and variants are tested in a single assay.

As used herein, the term “pathogen” refers to an organism such as, for example, an alga, an arachnid, a bacterium, a fungus, an insect, a nematode, a parasitic plant, yeast, a protozoan, or a virus capable of producing a disease in a plant or animal. The term “phytopathogen” as used herein refers to a pathogenic organism that infects a plant.

As used herein, “variant” in reference to a microorganism, is a strain having identifying characteristics of the species to which it belongs, while having at least one nucleotide sequence variation or identifiably different trait with respect to the parental strain, where the trait is genetically based (heritable). A nucleotide sequence variation includes substitutions, insertions, deletions or any combinations of such changes.

As used herein, seed treatment components are at least one pesticide or seed treatment formulation component as described herein and known in the art.

### High-Throughput Screen Methods

Microbial suspensions, which can be directly isolated from environmental samples, including for example soil or plant material, or from pooled samples of cultured microorganisms as described herein can be screened to identify microorganisms capable of surviving the conditions associated with plant seed treatment compositions and processes. Numerous media and buffers are known in the art that can be used in culturing, suspension and concentration steps in the described methods. In various embodiments, microbes may be applied on seeds, particularly crop plant seeds, or seed surrogates, including for example particles such as rocks, marbles, crystals, etc., or synthetic materials or surfaces.

In certain embodiments, the surrogate may be a seed of a different plant species. For instance, microorganisms associated with growing soybean plants may be tested for seed treatment tolerance in the methods herein by application to corn seeds. In some embodiments, selected microbial strains may be applied on a seed or seed surrogate as liquid coats or as dusts. Bacterial suspensions, for example, can be diluted in a solution and used directly to coat seeds. Alternatively, non-bacteria, for instance, can be applied to seeds as spore preparations. In another example, live cultures, fermentates, or liquid spore cultures can be applied to seeds. In yet another example, the treated seeds can be dried by tumbling and exposure to air. Microbes can also be mixed with inert carriers, dried, ground to a fine powder, and applied to seeds. Examples of inert carrier materials include talc, silica, fir bark, perlite, fluency agents, vermiculite, alginate, and clay.

In one embodiment, a pool of plant-associated microbes is applied to seeds at a pre-determined rate. Of particular interest is the application of large numbers of microbes to allow for efficient screening of diverse types of microbes in a single assay rather than the conventional screening method, which includes testing for survivability and viability on an individual isolate by isolate basis in a field or growth chamber/assay test (often a two year process). In a specific embodiment, the microorganism-treated seeds have a spore concentration or microbial cell concentration of from about 10⁶ to about 10⁹ per seed. In additional embodiments, the seeds may have more spores or microbial cells per seed, such as, for example 10¹⁰, 10¹¹ or 10¹² or more microbes or spores per seed. Concentrations can be readily adjusted, for example by measuring the turbidity and optical density of microbial cultures at 600 nm (OD₆₀₀), and applying the desired corresponding CFUs directly to the seeds.

Other seed treatments and formulation components as described herein can be applied prior to, concurrently with, or after the microbial treatment. For certain components, for example, polymers, application after the microbial treatment is applied may be preferred. In some embodiments, one or more pesticides (including fungicides, biocides, insecticides, herbicide, miticides, rodenticides, and nematicides), plant growth regulators (including LCOs, COs, chitinous compounds, flavonoids, jasmonic acid, methyl jasmonate, linoleic acid, linolenic acid, and karrikins) or micronutrients are also applied to the seeds. Application of seed treatment components may occur prior to, subsequent to or concurrent with application of the microorganisms. In one embodiment, microorganisms are applied to corn seeds that have been pre-treated with a composition comprising one or more pesticides selected from ipconazole, metalaxyl, trifloxystrobin, and clothianidin and optionally, one or more previously isolated beneficial microbes including, for example, Bacillus firmus. In another embodiment, microorganisms are applied to soybean seeds that have been pre-treated with a composition comprising one or more pesticides selected from pyraclostrobin, metalaxyl, fluxapyroxad and imidacloprid. In yet another embodiment, microorganisms are applied to cotton seeds that have been pre-treated with a composition comprising one or more pesticides selected from pyraclostrobin, metalaxyl, fluxapyroxad, myclobutanil, imidacloprid, ipconazole, thiamethoxam, chlorpyrifos, and abamectin. Various pesticides, plant growth regulators and micronutrients are known in the art and described herein and one skilled in the art can vary the composition and concentration of these compounds depending on, for example, the crop seed and desired beneficial effects.

In specific embodiments, the seeds or seed surrogates may be uniformly coated with microbial suspensions obtained according to the methods herein using conventional methods of mixing, spraying or a combination thereof through the use of treatment application equipment that is specifically designed and manufactured to accurately, safely, and efficiently apply seed treatment products to seeds.

A polymer-based seed finisher (“overcoat”) can optionally be added after or at the same time as application of microbes. The seeds are then stored under desired conditions for a period of time during which time some microorganisms are capable of being rendered inviable, enabling identification of at least a first microorganism remaining viable following storage. Storage conditions may be modified as needed to obtain the desired level and nature of stringency to identify microorganisms remaining viable following application of a given seed treatment. Non-limiting examples of storage conditions that may be varied include, for instance, storage time, temperature, and humidity. In one embodiment, storage is carried out under conditions typical for storing seeds prior to distribution and sale to farmers in the seed industry. In particular embodiments, storage is carried out from a range of less than an hour to a year or longer, including about one hour, about 6 hours, about 12 hours, about 24 hours, about 2 days, about a week, about two weeks, about a month, about two months, about 6 months and about a year or longer, and including all ranges derivable from these times. In other embodiments, storage is carried out at ambient temperature, or may be carried out at above or below ambient temperature, including about 0° C. or lower, about 4° C., about 25° C., about 37° C., and about 45° C. or higher, including all ranges derivable therefrom.

After the desired storage conditions, treated seeds can be soaked in a buffer to create a cell suspension which can be deposited, for example, onto solid agar growth media. The microbial colonies that form are derived from strains that survived the seed treatment and storage, and can be isolated, identified using microbial taxonomic methods, and archived for future use in field trials or other studies. Numerous media and buffers are known in the art and can be used in culturing, suspension and concentration steps in the described methods.

Of particular interest is the use in the methods described herein of various compounds used in commercial seed treatment formulations. Inclusion of such compounds allows for reproduction of commercial seed treatment conditions and ensures selection of microbes that will withstand such conditions under further testing and commercial applications. Further testing may include further screening under additional seed treatment conditions as well as testing in plant field conditions for microorganisms that can provide benefits to plants under commercial agricultural conditions.

Any plant seed capable of germinating to form a plant, including those described herein, that is susceptible to attack by nematodes, pathogenic fungi, and/or pathogenic bacteria can be treated with the microorganisms resulting from the methods described herein.

### Microorganisms

The method described herein is applicable to any microorganism, including any prokaryotic or eukaryotic microorganism. In some embodiments, the microorganism is a biological fungicide (“bf”) from at least one bacterium of the genus Actinomycetes, Agrobacterium, Arthrobacter, Alcaligenes, Aureobacterium, Azobacter, Bacillus, Beijerinckia, Brevibacillus, Burkholderia, Chromobacterium, Clostridium, Clavibacter, Comomonas, Corynebacterium, Curtobacterium, Enterobacter, Flavobacterium, Gluconobacter, Hydrogenophage, Klebsiella, Methylobacterium, Paenibacillus, Pasteuria, Phingobacterium, Photorhabdus, Phyllobacterium, Pseudomonas, Rhizobium, Serratia, Stenotrophomonas, Streptomyces, Variovorax, and Xenorhadbus. In particular embodiments the bacteria is selected from the group consisting of Bacillus amyloliquefaciens, Bacillus cereus, Bacillus firmus, Bacillus, lichenformis, Bacillus pumilus, Bacillus sphaericus, Bacillus subtilis, Bacillus thuringiensis, Pasteuria penetrans, Pasteuria usage, Pseudomona fluorescens, and combinations thereof.

In embodiments the biological fungicide (“bf”) can be a fungus of the genus Alternaria, Ampelomyces, Aspergillus, Aureobasidium, Beauveria, Candida, Colletotrichum, Coniothyrium, Cryphonectria, Fusarium, Gliocladium, Metarhizium, Metschnikowia, Microdochium, Muscodor, Paecilonyces, Phlebiopsis, Pseudozyma, Pythium, Trichoderma, Typhula, Ulocladium, and Verticilium. In particular embodiments the fungus is Beauveria bassiana, Coniothyrium minitans, Gliocladium vixens, Metarhizium anisopliae (also may be referred to in the art as Metarrhizium anisopliae, Metarhizium brunneum, or “green muscadine”), Muscodor albus, Paecilomyces lilacinus, Trichoderma polysporum, and combinations thereof.

Non-limiting examples of biological fungicides (“bf”) that may be suitable for use in the methods disclosed herein include Ampelomyces quisqualis (bf.1) (e.g., AQ 10® (bf.1a) from Intrachem Bio GmbH & Co. KG, Germany), Aspergillus flavus (bf.2) (e.g., AFLAGUARD® (bf.2a) from Syngenta, CH), Aureobasidium pullulans (bf.3) (e.g., BOTECTOR® (bf.3a) from bio-ferm GmbH, Germany), Bacillus pumilus (bf.4), Bacillus pumilus isolate AQ717, NRRL B-21662 (bf.4a) (from Fa. AgraQuest Inc., USA), Bacillus pumilus isolate NRRL B-30087 (bf.4b) (from Fa. AgraQuest Inc., USA), Bacillus sp., isolate AQ175, ATCC 55608 (bf.5) (from Fa. AgraQuest Inc., USA), Bacillus sp., isolate AQ177, ATCC 55609 (bf.6) (from Fa. AgraQuest Inc., USA), Bacillus subtilis (bf.7), Bacillus subtilis isolate AQ713, NRRL B-21661 (bf.7a) (in RHAPSODY®, SERENADE® MAX and SERENADE® ASO) (from Fa. AgraQuest Inc., USA), Bacillus subtilis isolate AQ743, NRRL B-21665 (bf.7b) (from Fa. AgraQuest Inc., USA), Bacillus amyloliquefaciens (bf.8) Bacillus amyloliquefaciens FZB24 (bf.8a) (e.g., TAEGRO® (bf.8b) from Novozymes Biologicals, Inc., USA), Bacillus amyloliquefaciens isolate NRRL B-50349 (bf.8c), Bacillus amyloliquefaciens TJ1000 (bf.8d) (i.e., also known as 1BE, isolate ATCC BAA-390), Bacillus thuringiensis (bf.9), Bacillus thuringiensis isolate AQ52, NRRL B-21619 (bf.9a) (from Fa. AgraQuest Inc., USA), Candida oleophila (bf.10), Candida oleophila 1-82 (bf.10a) (e.g., ASPIRE® (bf.10b) from Ecogen Inc., USA), Candida saitoana (bf.11) (e.g., BIOCURE® (bf.11a) (in mixture with lysozyme) and BIOCOAT® (bf.11b) from Micro Flo Company, USA (BASF SE) and Arysta), Clonostachys rosea f. catenulata, also named Gliocladium catenulatum (bf.12) (e.g., isolate J1446: PRESTOP® (bf.12a) from Verdera, Finland), Coniothyrium minitans (bf.13) (e.g., CONTANS® (bf.13a) from Prophyta, Germany), Cryphonectria parasitica (bf.14) (e.g., Endothia parasitica (bf.14a) from CNICM, France), Cryptococcus albidus (bf.15) (e.g., YIELD PLUS® (bf.15a) from Anchor Bio-Technologies, South Africa), Fusarium oxysporum (bf.16) (e.g., BIOFOX® (bf.16a) from S.I.A.P.A., Italy, FUSACLEAN® from Natural Plant Protection, France), Metschnikowia fructicola (bf.17) (e.g., SHEMER® (bf.17a) from Agrogreen, Israel), Microdochium dimerum (bf.18) (e.g., ANTIBOT® (bf.18a) from Agrauxine, France), Muscodor albus (bf.19), Muscodor albus isolate NRRL 30547 (bf.19a) (from Fa. AgraQuest Inc., USA), Muscodor roseus (bf.20), Muscodor roseus isolate NRRL 30548 (bf.20a) (from Fa. AgraQuest Inc., USA), Phlebiopsis gigantea (bf.21) (e.g., ROTSOP® (bf.21a) from Verdera, Finland), Pseudozyma flocculosa (bf.22) (e.g., SPORODEX® (bf.22a) from Plant Products Co. Ltd., Canada), Pythium oligandrum (bf.23), Pythium oligandrum DV74 (bf.23a) (e.g., POLYVERSUM® (bf.23b) from Remeslo SSRO, Biopreparaty, Czech Rep.), Talaromyces flavus (bf.24), Talaromyces flavus V117b (bf.24a) (e.g., PROTUS® (bf.24b) from Prophyta, Germany), Trichoderma asperellum (bf.25), Trichoderma asperellum SKT-1 (bf.25a) (e.g., ECO-HOPE® (bf.25b) from Kumiai Chemical Industry Co., Ltd., Japan), Trichoderma atroviride (bf.26), Trichoderma atroviride LC52 (bf.26a) (e.g., SENTINEL® (bf.26b) from Agrimm Technologies Ltd, NZ), Trichoderma harzianum (bf.27), Trichoderma harzianum T-22 (bf.27a) (e.g., PLANTSHIELD® (bf.27b) der Firma BioWorks Inc., USA), Trichoderma harzianum TH-35 (bf.27c) (e.g., ROOT PRO® (bf.27d) from Mycontrol Ltd., Israel), Trichoderma harzianum T-39 (bf.27e) (e.g., TRICHODEX® and TRICHODERMA 2000® (bf.27f) from Mycontrol Ltd., Israel and Makhteshim Ltd., Israel), Trichoderma harzianum ICC012 (bf.27g), Trichoderma harzianum and Trichoderma viride (bf.28) (e.g., TRICHOPEL (bf.28a) from Agrimm Technologies Ltd, NZ), Trichoderma harzianum ICC012 and Trichoderma viride ICC080 (bf.28b) (e.g., REMEDIER® WP (bf.28c) from Isagro Ricerca, Italy), Trichoderma polysporum and Trichoderma harzianum (bf.29) (e.g., BINAB® (bf.29a) from BINAB Bio-Innovation AB, Sweden), Trichoderma stromaticum (bf.30) (e.g., TRICOVAB® (bf.30a) from C.E.P.L.A.C., Brazil), Trichoderma virens (bf.31), Trichoderma virens GL-21 (bf.31a) (e.g., SOILGARD® (bf.31b) from Certis LLC, USA), Trichoderma virens G1-3 (bf.31c) (e.g., ATCC 58678, from Novozymes BioAg, Inc.), Trichoderma virens G1-21 (bf.31d) (commercially available from Thermo Trilogy Corporation), Trichoderma virens and Bacillus amyloliquefaciens (bf.32), Trichoderma virens G1-3 and Bacillus amyloliquefaciens FZB24 (bf.32a), Trichoderma virens G1-3 and Bacillus amyloliquefaciens isolate NRRL B-50349 (bf.32b), Trichoderma virens G1-3 and Bacillus amyloliquefaciens TJ1000 (bf.32c), Trichoderma virens G1-21 and Bacillus amyloliquefaciens FZB24 (bf.32d), Trichoderma virens G1-21 and Bacillus amyloliquefaciens isolate NRRL B-50349 (bf.32e), Trichoderma virens G1-21 and Bacillus amyloliquefaciens TJ1000 (bf.32f), Trichoderma viride (bf.33) (e.g., TRIECO® (bf.33a) from Ecosense Labs. (India) Pvt. Ltd., Indien, BIO-CURE® F from T. Stanes & Co. Ltd., Indien), Trichoderma viride TV1 (bf.33b) (e.g., Trichoderma viride TV1 from Agribiotec srl, Italy), Trichoderma viride ICC080 (bf.33c), Streptomyces sp. isolate NRRL No. B-30145 (bf.34) (from Fa. AgraQuest Inc., USA), Streptomyces sp. isolate M1064 (bf.35) (from Fa. AgraQuest Inc., USA), Streptomyces galbus (bf.36), Streptomyces galbus isolate NRRL 30232 (bf.36a) (from Fa. AgraQuest Inc., USA), Streptomyces lydicus (bf.37), Streptomyces lydicus WYEC 108 (bf.37a) (e.g., isolate ATCC 55445 in ACTINOVATE®, ACTINOVATE AG®, ACTINOVATE STP®, ACTINO-IRON®, ACTINOVATE L&G®, and ACTINOGROW® from Idaho Research Foundation, USA), Streptomyces violaceusniger (bf.38), Streptomyces violaceusniger YCED 9 (bf.38a) (e.g., isolate ATCC 55660 in DE-THATCH-9®, DECOMP-9®, and THATCH CONTROL® from Idaho Research Foundation, USA), Streptomyces WYE 53 (bf.39) (e.g., isolate ATCC 55750 in DE-THATCH-9®, DECOMP-9®, and THATCH CONTROL® from Idaho Research Foundation, USA) and Ulocladium oudemansii (bf.40), Ulocladium oudemansii HRU3 (bf.40a) (e.g., BOTRY-ZEN® (bf.40b) from Botry-Zen Ltd, NZ).

In a particular embodiment, the microorganism is a microbial insecticide, acaricide, or nematicide. Non-limiting examples of fungal insecticides, acaricides, or nematicides that may be used in the methods disclosed herein are described in McCoy, C. W., Samson, R. A., and Coucias, D. G. “Entomogenous fungi. In “CRC Handbook of Natural Pesticides. Microbial Pesticides, Part A. Entomogenous Protozoa and Fungi.” (C. M. Inoffo, ed.), (1988): Vol. 5, 151-236; Samson, R. A., Evans, H. C., and Latge′, J. P. “Atlas of Entomopathogenic Fungi.” (Springer-Verlag, Berlin) (1988); and deFaria, M. R. and Wraight, S. P. “Mycoinsecticides and Mycoacaricides: A comprehensive list with worldwide coverage and international classification of formulation types.” Biol. Control (2007), doi: 10.1016/j.biocontrol.2007.08.001.

In embodiments, the fungal insecticide, acaricide, or nematicide can be a fungus of the genus Aegerita, Akanthomyces, Alternaria, Arthrobotrys, Aschersonia, Ascophaera, Aspergillus, Beauveria, Blastodendrion, Calonectria, Coelemomyces, Coelomycidium, Conidiobolus, Cordyceps, Couchia, Culicinomyces, Dactylaria, Engyodontium, Entomophaga, Entomophthora, Erynia, Filariomyces, Filobasidiella, Fusarium, Gibellula, Harposporium, Hesperomyces, Hirsutella, Hymenostilbe, Hypocrella, Isaria, Lecanicillium, Lagenidium, Leptolegnia, Massospora, Metarhizium, Meristacrum, Metschnikowia, Monacrosporium, Mycoderma, Myiophagus, Myriangium, Myrothecium, Nectria, Nematoctonus, Neozygites, Nomuraea, Paecilomyces, Pandora, Paraisaria, Pasteuria, Pleurodesmospora, Pochonia, Podonectria, Polycephalomyces, Pseudogibellula, Septobasidium, Sorosporella, Sporodiniella, Stillbella, Tetranacrium, Tilachlidium, Tolypocladium, Torrubiella, Trenomyces, Trichoderma, Uredinella, Verticillium, Zoophthora, and combinations thereof.

Non-limiting examples of particular species that may be useful as a fungal insecticide, acaricide, or nematicide in the methods described herein include Alternaria cassia (mian.A1), Arthrobotrys dactyloides (mian.A2), Arthrobotrys oligospora (mian.A3), Arthrobotrys superb (mian.A4), Arthrobotrys dactyloides (mian.A5), Aspergillus parasiticus (mian.A6), Beauveria bassiana (mian.A7), Beauveria bassiana isolate ATCC-74040 (mian.A7a), Beauveria bassiana isolate ATCC-74250 (mian.A7b), Dactylaria candida (mian.A8), Fusarium lateritum (mian.A9), Fusarium solani (mian.A10), Harposporium anguillulae (mian.A11), Hirsutella rhossiliensis (mian.A12), Hirsutella minnesotensis (mian.A13), Lecanicillium lecanii (mian.A14), Metarhizium anisopliae (also may be referred to in the art as Metarrhizium anisopliae, Metarhizium brunneum, or “green muscadine”) (mian.A15), Metarhizium anisopliae isolate F52 (mian.A15a) (also known as Metarhizium anisopliae strain 52, Metarhizium anisopliae strain 7, Metarhizium anisopliae strain 43, Metarhizium anisopliae BIO-1020, TAE-001 and deposited as DSM 3884, DSM 3885, ATCC 90448, SD 170, and ARSEF 7711) (available from Novozymes Biologicals, Inc., USA)), Monacrosporium cionopagum (mian.A16), Nematoctonus geogenius (mian.A17), Nematoctonus leiosporus (mian.A18), Meristacrum asterospermum (mian.A19), Myrothecium verrucaria (mian.A20), Paecilomyces fumosoroseus (mian.A21), Paecilomyces fumosoroseus FE991 (mian.A21a) (in NOFLY® from FuturEco BioScience S.L., Barcelona, Spain), Paecilomyces lilacinus (mian.A22), Pasteuria penetrans (mian.A23), Pasteuria usage (mian.A24), Pochonia chlamydopora (mian.A25), Trichoderma hamatum (mian.A26), Trichoderma harzianum (mian.A27), Trichoderma vixens (mian.A28), Verticillium chlamydosporum (mian.A29), Verticillium lecanii (mian.A30), and combinations thereof.

In a particular embodiment, the microorganism is a bacterial insecticide, acaricids, or nematicide. In embodiments, the bacterial insecticide, acaricide, or nematicide can be a bacterium of the genus Actinomycetes Agrobacterium, Arthrobacter, Alcaligenes, Aureobacterium, Azobacter, Bacillus, Beijerinckia, Burkholderia, Chromobacterium, Clavibacter, Clostridium, Comomonas, Corynebacterium, Curtobacterium, Desulforibtio, Enterobacter, Flavobacterium, Gluconobacter, Hydrogenophage, Klebsiella, Methylobacterium, Paenibacillus, Phyllobacterium, Phingobacterium, Photorhabdus, Pseudomonas, Rhodococcus, Serratia, Stenotrotrophomonas, Streptomyces, Xenorhadbus, Variovorax, and combinations thereof. Non-limiting examples of particular species that may be useful as a bacterial insecticide, acaricide, or nematicide in the methods described herein include Bacillus firmus (mian.B1), Bacillus firmus isolate 1-1582 (mian.B1a) (in BioNeem, Votivo), Bacillus mycoides (mian.B2), Bacillus mycoides isolate AQ726, NRRL B-21664 (mian.B2a), Burkholderia sp. (mian.B3), Burkholderia sp. nov. rinojensis (mian.B3a), Burkholderia sp. A396 sp. nov. rinojensis, NRRL B-50319 (mian.B3b), Chromobacterium subtsugae (mian.B4), Chromobacterium subtsugae sp. nov. (mian.B4a), Chromobacterium subtsugae sp. nov. isolate NRRL B-30655 (mian.B4b), Chromobacterium vaccinii (mian.B5), Chromobacterium vaccinii isolate NRRL B-50880 (mian.B5a), Chromobacterium violaceum (mian B6), Flavobacterium sp. (mian.B7), Flavobacterium sp. isolate H492, NRRL B-50584 (mian B7a), Streptomyces lydicus (mian B8), Streptomyces violaceusniger (mian B9), and combinations thereof.

In one embodiment, the methods described herein may further comprise at least one beneficial microorganism (“bm”). The at least one beneficial microorganism may be in a spore form, a vegetative form, or a combination thereof.

In particular embodiments, the at least one beneficial microorganism (“bm”) is a diazotroph (i.e., bacteria which are symbiotic nitrogen-fixing bacteria). In embodiments, the diazotroph is a bacterium of the genus Azorhizobium, Azospirillum, Bradyrhizobium, Mesorhizobium, Rhizobium, Sinorhizobium, and combinations thereof. Non-limiting examples of particular species that may be useful as a bacterial diazotroph in the methods described herein include Azorhizobium caulinodans (bm.A1), Azorhizobium doebereinerae (bm.A2), Azospirillum amazonense (bm.A3), Azospirillum brasilense (bm.A4), Azospirillum brasilense isolate INTA Az-39 (bm.A4a) (available from Novozymes), Azospirillum canadense (bm.A5), Azospirillum doebereinerae (bm.A6), Azospirillum formosense (bm.A7), Azospirillum halopraeferans (bm.A8), Azospirillum irakense (bm.A9), Azospirillum largimobile (bm.A10), Azospirillum lipoferum (bm.A11), Azospirillum melinis (bm.A12), Azospirillum oryzae (bm.A13), Azospirillum picis (bm.A14), Azospirillum rugosum (bm.A15), Azospirillum thiophilum (bm.A16), Azospirillum zeae (bm.A17), Bradyrhizobium bete (bm.A18), Bradyrhizobium canadense (bm.A19), Bradyrhizobium elkanii (bm.A20), Bradyrhizobium elkanii isolate SEMIA 587 (bm.A20a) (available from Novozymes), Bradyrhizobium elkanii isolate SEMIA 5019 (bm.A20b) (available from Novozymes), Bradyrhizobium iriomotense (bm.A21), Bradyrhizobium japonicum (bm.A22), Bradyrhizobium japonicum isolate SEMIA 5079 (bm.A22a) (available from Novozymes), Bradyrhizobium japonicum isolate SEMIA 5080 (bm.A22b) (available from Novozymes), Bradyrhizobium japonicum isolate NRRL B-50608 (bm.A22c) (available from Novozymes), Bradyrhizobium japonicum isolate NRRL B-50609 (bm.A22d) (available from Novozymes), Bradyrhizobium japonicum isolate NRRL B-50610 (bm.A22e) (available from Novozymes), Bradyrhizobium japonicum isolate NRRL B-50611 (bm.A22f) (available from Novozymes), Bradyrhizobium japonicum isolate NRRL B-50612 (bm.A22g) (available from Novozymes), Bradyrhizobium japonicum isolate NRRL B-50592 (deposited also as NRRL B-59571) (bm.A22h) (available from Novozymes), Bradyrhizobium japonicum isolate NRRL B-50593 (deposited also as NRRL B-59572) (bm.A22i) (available from Novozymes), Bradyrhizobium japonicum isolate NRRL B-50586 (deposited also as NRRL B-59565) (bm.A22j) (available from Novozymes), Bradyrhizobium japonicum isolate NRRL B-50588 (deposited also as NRRL B-59567) (bm.A22k) (available from Novozymes), Bradyrhizobium japonicum isolate NRRL B-50587 (deposited also as NRRL B-59566) (bm.A22l) (available from Novozymes), Bradyrhizobium japonicum isolate NRRL B-50589 (deposited also as NRRL B-59568) (bm.A22m) (available from Novozymes), Bradyrhizobium japonicum isolate NRRL B-50591 (deposited also as NRRL B-59570) (bm.A22n) (available from Novozymes), Bradyrhizobium japonicum NRRL B-50590 (deposited also as NRRL B-59569) (bm.A22o) (available from Novozymes), Bradyrhizobium japonicum isolate NRRL B-50594 (deposited also as NRRL B-50493) (bm.A22p) (available from Novozymes), Bradyrhizobium japonicum isolate NRRL B-50726 (bm.A22q) (available from Novozymes), Bradyrhizobium japonicum isolate NRRL B-50727 (bm.A22r) (available from Novozymes), Bradyrhizobium japonicum isolate NRRL B-50728 (bm.A22s) (available from Novozymes), Bradyrhizobium japonicum isolate NRRL B-50729 (bm.A22t) (available from Novozymes), Bradyrhizobium japonicum isolate NRRL B-50730 (bm.A22u) (available from Novozymes), Bradyrhizobium japonicum isolate USDA 532C (bm.A22v), Bradyrhizobium japonicum isolate USDA 110 (bm.A22w), Bradyrhizobium japonicum isolate USDA 123 (bm.A22x), Bradyrhizobium japonicum isolate USDA 127 (bm.A22y), Bradyrhizobium japonicum isolate USDA 129 (bm.A22z), Bradyrhizobium jicamae (bm.A23), Bradyrhizobium liaoningense (bm.A24), Bradyrhizobium pachyrhizi (bm.A25), Bradyrhizobium yuanmingense (bm.A26), Mesorhizobium albiziae (bm.A27), Mesorhizobium amorphae (bm.A28), Mesorhizobium chacoense (bm.A29), Mesorhizobium ciceri (bm.A30), Mesorhizobium huakuii (bm.A31), Mesorhizobium loti (bm.A32), Mesorhizobium mediterraneum (bm.A33), Mesorhizobium pluifarium (bm.A34), Mesorhizobium septentrionale (bm.A35), Mesorhizobium temperatum (bm.A36), Mesorhizobium tianshanense (bm.A37), Rhizobium cellulosilyticum (bm.A38), Rhizobium daejeonense (bm.A39), Rhizobium etli (bm.A40), Rhizobium galegae (bm.A41), Rhizobium gallicum (bm.A42), Rhizobium giardinii (bm.A43), Rhizobium hainanense (bm.A44), Rhizobium huautlense (bm.A45), Rhizobium indigoferae (bm.A46), Rhizobium leguminosarum (bm.A47), Rhizobium leguminosarum isolate SO12A-2-(IDAC 080305-01) (bm.A47a), Rhizobium loessense (bm.A48), Rhizobium lupini (bm.A49), Rhizobium lusitanum (bm.A50), Rhizobium meliloti (bm.A51), Rhizobium mongolense (bm.A52), Rhizobium miluonense (bm.A53), Rhizobium sullae (bm.A54), Rhizobium tropici (bm.A55), Rhizobium undicola (bm.A56), Rhizobium yanglingense (bm.A57), Sinorhizobium abri (bm.A58), Sinorhizobium adhaerens (bm.A59), Sinorhizobium americanum (bm.A60), Sinorhizobium aboris (bm.A61), Sinorhizobium fredii (bm.A62), Sinorhizobium indiaense (bm.A63), Sinorhizobium kostiense (bm.A64), Sinorhizobium kummerowiae (bm.A65), Sinorhizobium medicae (bm.A66), Sinorhizobium meliloti (bm.A67), Sinorhizobium mexicanus (bm.A68), Sinorhizobium morelense (bm.A69), Sinorhizobium saheli (bm.A70), Sinorhizobium terangae (bm.A71), Sinorhizobium xinjiangense (bm.A72), and combinations thereof.

In particular embodiments, the at least one beneficial microorganism (“bm”) is a phosphate solubilizing microorganism. In embodiments, the at least one phosphate solubilizing microorganism is a fungus of the genus Penicillium, Talaromyces, and combinations thereof. Non-limiting examples of particular species that may be useful as a phosphate solubilizing fungus in the methods described herein include Penicillium albidum (bm.B1), Penicillium aurantiogriseum (bm.B2), Penicillium bilaiae (formerly known as Penicillium bilaii and Penicillium bilaji) (bm.B3), Penicillium bilaiae isolate ATCC 20851 (bm.B3a), Penicillium bilaiae isolate ATCC 22348 (bm.B3b), Penicillium bilaiae isolate V08/021001 (also deposited as NRRL B-50612) (bm.B3c), Penicillium bilaiae isolate NRRL B-50776 (bm.B3d), Penicillium bilaiae isolate NRRL B-50777 (bm.B3e), Penicillium bilaiae isolate NRRL B-50778 (bm.B3f), Penicillium bilaiae isolate NRRL B-50779 (bm.B3g), Penicillium bilaiae isolate NRRL B-50780 (bm.B3h), Penicillium bilaiae isolate NRRL B-50781 (bm.B3i), Penicillium bilaiae isolate NRRL B-50782 (bm.B3j), Penicillium bilaiae isolate NRRL B-50783 (bm.B3k), Penicillium bilaiae isolate NRRL B-50784 (bm.B3l), Penicillium bilaiae isolate NRRL B-50785 (bm.B3m), Penicillium bilaiae isolate NRRL B-50786 (bm.B3n), Penicillium bilaiae isolate NRRL B-50787 (bm.B3o), Penicillium bilaiae isolate NRRL B-50788 (bm.B3p), Penicillium bilaiae isolate NRRL B-50169 (bm.B3q), Penicillium bilaiae isolate ATCC 18309 (bm.B3r), Penicillium brevicompactum (bm.B4), Penicillium brevicompactum isolate AgRF18 (bm.B4a), Penicillium canescens (bm.B5), Penicillium canescens isolate ATCC 10419 (bm.B5a), Penicillium chrysogenum (bm.B6), Penicillium citreonigrum (bm.B7), Penicillium citrinum (bm.B8), Penicillium digitatum (bm.B9), Penicillium expansum (bm.B10), Penicillium expansum isolate ATCC 24692 (bm.B10a), Penicillium expansum isolate YT02 (bm.B10b), Penicillium fellutanum (bm.B11), Penicillium fellutanum isolate ATCC 48694 (bm.B11a), Penicillium frequentas (bm.B12), Penicillium fuscum (bm.B13), Penicillium fussiporus (bm.B14), Penicillium gaestrivorus (bm.B15), Penicillium gaestrivorus isolate NRRL 50170 (bm.B15a), Penicillium glabrum (bm.B16), Penicillium glabrum isolate DAOM 239074 (bm.B16a), Penicillium glabrum isolate CBS 229.28 (bm.B16b), Penicillium glaucum (bm.B17), Penicillium griseofulvum (bm.B18), Penicillium implicatum (bm.B19), Penicillium janthinellum (bm.B20), Penicillium janthinellum isolate ATCC 10455 (bm.B20a), Penicillium lanosocoeruleum (bm.B21), Penicillium lanosocoeruleum isolate ATCC 48919 (bm.B21a), Penicillium lilacinum (bm.B22), Penicillium minioluteum (bm.B23), Penicillium montanense (bm.B24), Penicillium nigricans (bm.B25), Penicillium oxalicum (bm.B26), Penicillium pinetorum (bm.B27), Penicillium pinophilum (bm.B28), Penicillium purpurogenum (bm.B29), Penicillium radicum (bm.B30), Penicillium radicum isolate N93/47267 (bm.B30a), Penicillium radicum isolate FRR 4717 (bm.B30b), Penicillium radicum isolate ATCC 201836 (bm.B30c), Penicillium radicum isolate FRR 4719 (bm.B30d), Penicillium raistrickii (bm.B31), Penicillium raistrickii isolate ATCC 10490 (bm.B31a), Penicillium rugulosum (bm.B32), Penicillium simplicissimum (bm.B33), Penicillium solitum (bm.B34), Penicillium variabile (bm.B35), Penicillium velutinum (bm.B36), Penicillium viridicatum (bm.B37), Talaromyces aculeatus (bm.B38), Talaromyces aculeatus isolate ATCC 10409 (bm.B38a), and combinations thereof.

In particular embodiments, the at least one beneficial microorganism (“bm”) is a mycorrhiza. Suitable mycorrhizae include endomycorrhiza (also called vesicular arbuscular mycorrhiza, VAMs, arbuscular mycorrhiza, or AMs), ectomycorrhiza, ericoid mycorrhiza, and combinations thereof. In embodiments, the mycorrhiza is a fungus of the genus Gigaspora, Glomus, Hymenoscyphous, Laccaria, Oidiodendron, Paraglomus, Pisolithus, Rhizoctonia, Rhizopogon, Scleroderma, and combinations thereof. Non-limiting examples of particular mycorrhizal species that may be useful in the compositions described herein include Gigaspora margarita (bm.C1), Glomus aggregatum (bm.C2), Glomus brasilianum (bm.C3), Glomus clarum (bm.C4), Glomus deserticola (bm.C5), Glomus etunicatum (bm.C6), Glomus fasciculatum (bm.C7), Glomus intraradices (bm.C8), Glomus monosporum (bm.C9), Glomus mosseae (bm.C10), Hymenoscyphous ericae (bm.C11), Laccaria bicolor (bm.C12), Laccaria laccata (bm.C13), Oidiodendron sp. (bm.C14), Paraglomus brazilianum (bm.C15), Pisolithus tinctorius (bm.C16), Rhizoctonia sp. (bm.C17), Rhizopogon amylopogon (bm.C18), Rhizopogon fulvigleba (bm.C19), Rhizopogon luteolus (bm.C20), Rhizopogon villosuli (bm.C21), Scleroderma cepa (bm.C22), Scleroderma citrinum (bm.C23), Rhizoplex® (Gigaspora margarita, Glomus aggregatum, Glomus brasilianum, Glomus clarum, Glomus deserticola, Glomus etunicatum, Glomus intraradices, Glomus monosporum, Glomus mosseae, Laccaria bicolor, Laccaria laccata, Pisolithus tinctorius, Rhizopogon amylopogon, Rhizopogon fulvigleba, Rhizopogon luteolus, Rhizopogon villosuli, Scleroderma cepa and Scleroderma citrinum) (bm.C24) (available from Novozymes), Rhizomyco® (Gigaspora margarita, Glomus aggregatum, Glomus clarum, Glomus deserticola, Glomus etunicatum, Glomus intraradices, Glomus monosporum, Glomus mosseae, Laccaria bicolor, Laccaria laccata, Paraglomus brazilianum, Pisolithus tinctorius, Rhizopogon amylopogon, Rhizopogon fulvigleba, Rhizopogon luteolus, Rhizopogon villosuli, Scleroderma cepa and Scleroderma citrinum) (bm.C25) (available from Novozymes), Rhizomyx® (Gigaspora margarita, Glomus aggregatum, Glomus brasilianum, Glomus clarum, Glomus deserticola, Glomus etunicatum, Glomus intraradices, Glomus monosporum, and Glomus mosseae) (bm.C26) (available from Novozymes), and combinations thereof.

Microorganisms to be screened for ability to survive seed treatment components and processes in the methods provided herein can be obtained from any number of environments. In one embodiment, the microorganisms are obtained from environmental soil samples, including soil obtained from non-agricultural sites or soil from sites currently or previously used for agriculture, for example for commercial or small scale farming. When soil samples are obtained from minimally disturbed non-agricultural sites, for example, the methods described herein may result in isolation, selection, and identification of microbes that are foreign to modern agricultural settings. Soils can be obtained from any environment that supports the growth of microorganisms, and can include, for example, sandy soils, clay soils, prairie soils, peaty soils, and loamy soils.

In one embodiment, the microorganisms tested in the methods described herein are found associated with a given plant of interest prior to testing for tolerance of seed treatment components and methods. For example, microbes may be associated with crop plants growing in an agricultural field, i.e., a field in which crop plants are cultivated, as distinguished from an uncultivated environment in which crop plants are not cultivated. Alternatively, soil samples from an environment of interest may be collected and crop plant seeds planted in the soil and allowed to grow in a controlled environment, including for example, plant growth chambers or greenhouses. Where plants are grown in a controlled environment, the environmental soil sample can be mixed with inorganic or organic plant growth materials to optimize plant growth conditions, including such materials as vermiculite, perlite, peat and the like. Whether the plants are grown in agricultural fields or controlled environments, the plants and rhizosphere can be collected and the associated microorganisms extracted from the samples. The plants and soil may be collected at any number of plant growth stages, but preferentially, the plants are harvested after appearance of the first true leaf. Plants may also be harvested at later growth stages including, for example early vegetative stage, (V3-V4), late vegetative stage, or even at maturity.

The initial source of plant associated microorganisms does not limit the plants that may benefit from application of the identified microorganisms. For example, microorganisms associated with one plant species may be shown to provide beneficial properties for growth of crop plants from the same or a different species.

In one embodiment, rhizospheric microbial cell suspensions may be produced from crop plant roots and microorganisms isolated therefrom, for example by agitating the plant materials and/or cell suspension (e.g., sonication) to liberate root-associated microbes into the solution. In other embodiments, an endospheric/phyllospheric cell suspension can be produced by submerging plant shoots, stems, leaves, flowers or fruit, into a solution and macerating them to a pulp consistency by vigorous blending and/or bead beating. In certain embodiments, a microbial cell suspension is concentrated to increase the number of microorganisms per volume of the preparation. In one embodiment, a microbial suspension for use in the methods herein is concentrated to provide a suspension having from 10⁶ to 10⁹ CFUs per milliliter of suspension. In yet other embodiments, resulting microbial cell suspensions are purified using methods known to those of skill in the art (e.g., filtration, differential and/or density-gradient centrifugation) to remove plant/soil debris and provide more concentrated microbial suspensions. In one embodiment, further purification is used to provide pooled bacterial suspensions from multiple plants, including plants of the same or different species. In yet other embodiments, the cell suspension can be a representative sample of all of the bacteria, fungi, and archaea present in a microbiota, including, but not limited to, those found in soils, plant tissues, and bodies of water. In other embodiments using cultured microorganisms, collections of known microbes, from culture collections, for example, may be produced as described herein or as known in the art, whether previously associated with growing plants or simply isolated from an environmental sample, and screened using the methods described herein.

### Seed Treatment Components

The seed treatment components in the screening methods described herein may comprise at least one pesticide. The seed treatment may include, for example, a fungicide (“f”). Useful fungicides may be biological fungicides (“bf”), chemical fungicides (“cf”), or combinations thereof. Fungicides may be selected so as to be provide effective control against a broad spectrum of phytopathogenic fungi, including soil-borne fungi, which derive especially from the classes of the Plasmodiophoromycetes, Peronosporomycetes (syn. Oomycetes), Chytridiomycetes, Zygomycetes, Ascomycetes, Basidiomycetes, and Deuteromycetes (syn. Fungi imperfecti). More common fungal pathogens that may be effectively targeted include Pytophthora, Rhizoctonia, Fusarium, Pythium, Phomopsis or Selerotinia and Phakopsora and combinations thereof.

In certain embodiments, the fungicide is a chemical fungicide (“cf”). Representative examples of useful chemical fungicides (“cf”) that may be suitable for use in the present disclosure include aromatic hydrocarbons, benzimidazoles, benzthiadiazole, carboxamides, carboxylic acid amides, morpholines, phenylamides, phosphonates, quinone outside inhibitors (e.g., strobilurins), thiazolidines, thiophanates, thiophene carboxamides, and triazoles:

A) strobilurins (cf.A):

azoxystrobin (cf.A1), coumethoxystrobin (cf.A2), coumoxystrobin (cf.A3), dimoxystrobin (cf.A4), enestroburin (cf.A5), fluoxastrobin (cf.A6), kresoxim-methyl (cf.A7), metominostrobin (cf.A8), orysastrobin (cf.A9), picoxystrobin (cf.A10), pyraclostrobin (cf.A11), pyrametostrobin (cf.A12), pyraoxystrobin (cf.A13), pyribencarb (cf.A14), trifloxystrobin (cf.A15), 2-[2-(2,5-dimethyl-phenoxymethyl)-phenyl]-3-methoxy-acrylic acid methyl ester (cf.A16), and 2-(2-(3-(2,6-dichlorophenyl)-1-methyl-allylideneaminooxymethyl)-phenyl)-2-methoxyimino-N-methyl-acetamide (cf.A17);

B) carboxamides (cf.B):

carboxanilides (cf.B1): benalaxyl (cf.B1a), benalaxyl-M (cf.B1b), benodanil (cf.B1c), bixafen (cf.B1d), boscalid (cf.B1d), carboxin (cf.B1e), fenfuram (cf.B1f), fenhexamid (cf.B1g), flutolanil (cf.B1h), fluxapyroxad (cf.B1i), furametpyr (cf.B1j), isopyrazam (cf.B1k), isotianil (cf.B1l), kiralaxyl (cf.B1m), mepronil (cf.B1n), metalaxyl (cf.B1o), metalaxyl-M (mefenoxam) (cf.B1p), ofurace (cf.B1q), oxadixyl (cf.B1r), oxycarboxin (cf.B1s), penflufen (cf.B1t), penthiopyrad (cf.B1u), sedaxane (cf.B1v), tecloftalam (cf.B1w), thifluzamide (cf.B1x), tiadinil (cf.B1y), 2-amino-4-methyl-thiazole-5-carboxanilide (cf.B1z), N-(4′-trifluoromethylthiobiphenyl-2-yl)-3-difluoromethyl-1-methyl-1H-pyra-zole-4-carboxamide (cf.B1aa), and N-(2-(1,3,3-trimethylbutyl)-phenyl)-1,3-dimethyl-5-fluoro-1H-pyrazole-4-carboxamide (cf.B1ab);

carboxylic morpholides (cf.B2): dimethomorph (cf.B2a), flumorph (cf.B2b), pyrimorph (cf.B2c); benzoic acid amides (cf.B3): flumetover (cf.B3a), fluopicolide (cf.B3b), fluopyram (cf.B3c), zoxamide (cf.B3d); other carboxamides (cf.B4): carpropamid (cf.B4a), dicyclomet (cf.B4b), mandiproamid (cf.B4c), oxytetracyclin (cf.B4d), silthiofam (cf.B4e), and N-(6-methoxy-pyridin-3-yl) cyclopropanecarboxylic acid amide (cf.B4f);

C) azoles (cf.C):

triazoles (cf.C1): azaconazole (cf.C1a), bitertanol (cf.C1b), bromuconazole (cf.C1c), cyproconazole (cf.C1d), difenoconazole (cf.C1e), diniconazole (cf.C1f), diniconazole-M (cf.C1g), epoxiconazole (cf.C1h), fenbuconazole (cf.C1i), fluquinconazole (cf.C1j), flusilazole (cf.C1k), flutriafol (cf.C1l), hexaconazole (cf.C1m), imibenconazole (cf.C1n), ipconazole (cf.C1o), metconazole (cf.C1p), myclobutanil (cf.C1q), oxpoconazole (cf.C1r), paclobutrazole (cf.C1s), penconazole (cf.C1t), propiconazole (cf.C1u), prothioconazole (cf.C1v), simeconazole (cf.C1w), tebuconazole (cf.C1x), tetraconazole (cf.C1y), triadimefon (cf.C1z), triadimenol (cf.C1aa), triticonazole (cf.C1ab), uniconazole (cf.C1ac); imidazoles (cf.C2): cyazofamid (cf.C2a), imazalil (cf.C2b), pefurazoate (cf.C2c), prochloraz (cf.C2d), triflumizol (cf.C2e);

D) heterocyclic compounds (cf.D):

pyridines (cf.D1): fluazinam (cf.D1a), pyrifenox (cf.D1b), 3-[5-(4-chloro-phenyl)-2,3-dimethyl-isoxazolidin-3-yl]-pyridine (cf.D1c), 3-[5-(4-methyl-phenyl)-2,3-dimethyl-isoxazolidin-3-yl]-pyridine (cf.D1d); pyrimidines (cf.D2): bupirimate (cf.D2a), cyprodinil (cf.D2b), diflumetorim (cf.D2c), fenarimol (cf.D2d), ferimzone (cf.D2e), mepanipyrim (cf.D2f), nitrapyrin (cf.D2g), nuarimol (cf.D2h), pyrimethanil (cf.D2i); piperazines (cf.D3): triforine (cf.D3a); pyrroles (cf.D4): fenpiclonil (cf.D4a), fludioxonil (cf.D4b); morpholines (cf.D5): aldimorph (cf.D5a), dodemorph (cf.D5b), dodemorph-acetate (cf.D5c), fenpropimorph (cf.D5d), tridemorph (cf.D5e); piperidines (cf.D6): fenpropidin (cf.D6a); dicarboximides (cf.D7): fluoroimid (cf.D7a), iprodione (cf.D7b), procymidone (cf.D7c), vinclozolin (cf.D7d); non-aromatic 5-membered heterocycles (cf.D8): famoxadone (cf.D8a), fenamidone (cf.D8b), flutianil (cf.D8c), octhilinone (cf.D8d), probenazole (cf.D8e), 5-amino-2-isopropyl-3-oxo-4-ortho-tolyl-2,3-dihydro-pyrazole-1-carbothioic acid S-allyl ester (cf.D8f); others (cf.D9): acibenzolar-S-methyl (cf.D9a), ametoctradin (cf.D9b), amisulbrom (cf.D9c), anilazin (cf.D9d), blasticidin-S (cf.D9e), captafol (cf.D9f), captan (cf.D9g), chinomethionat (cf.D9h), dazomet (cf.D9i), debacarb (cf.D9j), diclomezine (cf.D9k), difenzoquat (cf.D9l), difenzoquat-methylsulfate (cf.D9m), fenoxanil (cf.D9n), Folpet (cf.D9o), oxolinic acid (cf.D9p), piperalin (cf.D9q), proquinazid (cf.D9r), pyroquilon (cf.D9s), quinoxyfen (cf.D9t), triazoxide (cf.D9u), tricyclazole (cf.D9v), 2-butoxy-6-iodo-3-propylchromen-4-one (cf.D9w), 5-chloro-1-(4,6-dimethoxy-pyrimidin-2-yl)-2-methyl-1H-benzoimidazole (cf.D9x), and 5-chloro-7-(4-methylpiperidin-1-yl)-6-(2,4,6-trifluorophenyl)-[1,2,4]triazolo-[1,5-a]pyrimidine (cf.D9y);

E) benzimidazoles (cf.E): carbendazim (cf.E1).

F) other active substances (cf.F):

guanidines (cf.F1): guanidine (cf.F1a), dodine (cf.F1b), dodine free base (cf.F1c), guazatine (cf.F1d), guazatine-acetate (cf.F1e), iminoctadine (cf.F1f), iminoctadine-triacetate (cf.F1g), iminoctadine-tris(albesilate) (cf.F1h); antibiotics (cf.F2): kasugamycin (cf.F2a), kasugamycin hydrochloride-hydrate (cf.F2b), streptomycin (cf.F2c), polyoxine (cf.F2d), validamycin A (cf.F2e); nitrophenyl derivates (cf.F3): binapacryl (cf.F3a), dicloran (cf.F3b), dinobuton (cf.F3c), dinocap (cf.F3d), nitrothal-isopropyl (cf.F3e), tecnazen (cf.F3f), organometal compounds (cf.F4): fentin salts (cf.F4a), such as fentin-acetate (cf.F4b), fentin chloride (cf.F4c), or fentin hydroxide (cf.F4d); sulfur-containing heterocyclyl compounds (cf.F5): dithianon (cf.F5a), isoprothiolane (cf.F5b); organophosphorus compounds (cf.F6): edifenphos (cf.F6a), fosetyl (cf.F6b), fosetyl-aluminum (cf.F6c), iprobenfos (cf.F6d), phosphorus acid and its salts (cf.F6e), pyrazophos (cf.F6f), tolclofos-methyl (cf.F6g); organochlorine compounds (cf.F7): chlorothalonil (cf.F7a), dichlofluanid (cf.F7b), dichlorophen (cf.F7c), flusulfamide (cf.F7d), hexachlorobenzene (cf.F7e), pencycuron (cf.F7f), pentachlorphenole and its salts (cf.F7g), phthalide (cf.F7h), quintozene (cf.F7i), thiophanate-methyl (cf.F7j), thiophanate (cf.F7k), tolylfluanid (cf.F7l), N-(4-chloro-2-nitro-phenyl)-N-ethyl-4-methyl-benzenesulfonamide (cf.F7m); inorganic active substances (cf.F8): Bordeaux mixture (cf.F8a), copper acetate (cf.F8b), copper hydroxide (cf.F8c), copper oxychloride (cf.F8d), basic copper sulfate (cf.F8e), and sulfur (cf.F8f).

Commercial products containing fungicides are readily available. Fungicide concentration in the method will generally correspond to the labeled use rate for a particular fungicide.

In one embodiment, the seed treatment components used in the methods described herein may include at least one herbicide (“h”).

Non-limiting examples of types of herbicides include acetyl CoA carboxylase (ACCase) inhibitors (h.A), acetolactate synthase (ALS) (h.B) or acetohydroxy acid synthase (AHAS) inhibitors (h.C), photosystem II inhibitors (h.D), photosystem I inhibitors (h.E), protoporphyrinogen oxidase (PPO or Protox) inhibitors (h.F), carotenoid biosynthesis inhibitors (h.G), enolpyruvyl shikimate-3-phosphate (EPSP) synthase inhibitor (h.H), glutamine synthetase inhibitor (h.I), dihydropteroate synthetase inhibitor (h.J), mitosis inhibitors (h.K), 4-hydroxyphenyl-pyruvate-dioxygenase (4-HPPD) inhibitors (h.L), synthetic auxins (h.M), auxin herbicide salts (h.N), auxin transport inhibitors (h.O), and nucleic acid inhibitors (h.P), salts and esters thereof; racemic mixtures and resolved isomers thereof; and combinations thereof.

Specific examples of possible herbicides (“h”) that may be used in seed treatment methods described herein include 2,4-dichlorophenoxyacetic acid (2,4-D) (h.1), 2,4,5-trichlorophenoxyacetic acid (2,4,5-T) (h.2), ametryn (h.3), amicarbazone (h.4), aminocyclopyrachlor (h.5), acetochlor (h.6), acifluorfen (h.7), alachlor (h.8), atrazine (h.9), azafenidin (h.10), bentazon (h.11), benzofenap (h.12), bifenox (h.13), bromacil (h.14), bromoxynil (h.15), butachlor (h.16), butafenacil (h.17), butroxydim (h.18), carfentrazone-ethyl (h.19), chlorimuron (h.20), chlorotoluron (h.21), clethodim (h.22), clodinafop (h.23), clomazone (h.24), cyanazine (h.25), cycloxydim (h.26), cyhalofop (h.27), desmedipham (h.28), desmetryn (h.29), dicamba (h.30), diclofop (h.31), dimefuron (h.32), diuron (h.33), dithiopyr (h.34), fenoxaprop (h.35), fluazifop (h.36), fluazifop-P (h.37), fluometuron (h.38), flufenpyr-ethyl (h.39), flumiclorac-pentyl (h.40), flumioxazin (h.41), fluoroglycofen (h.42), fluthiacet-methyl (h.43), fomesafe (h.44), fomesafen (h.45), glyphosate (h.46), glufosinate (h.47), haloxyfop (h.48), hexazinone (h.49), imazamox (h.50), imazaquin (h.51), imazethapyr (h.52), ioxynil (h.53), isoproturon (h.54), isoxaflutole (h.55), lactofen (h.56), linuron (h.57), mecoprop (h.58), mecoprop-P (h.59), mesotrione (h.60), metamitron (h.61), metazochlor (h.62), methibenzuron (h.63), metolachlor (h.64) (and S-metolachlor (h.65)), metoxuron (h.66), metribuzin (h.67), monolinuron (h.68), oxadiargyl (h.69), oxadiazon (h.70), oxyfluorfen (h.71), phenmedipham (h.72), pretilachlor (h.73), profoxydim (h.74), prometon (h.75), prometryn (h.76), propachlor (h.77), propanil (h.78), propaquizafop (h.79), propisochlor (h.80), pyraflufen-ethyl (h.81), pyrazon (h.82), pyrazolynate (h.83), pyrazoxyfen (h.84), pyridate (h.85), quizalofop (h.86), quizalofop-P (h.87) (e.g., quizalofop-ethyl (h.88), quizalofop-P-ethyl (h.89), clodinafop-propargyl (h.90), cyhalofop-butyl (h.91), diclofop-methyl (h.92), fenoxaprop-P-ethyl (h.93), fluazifop-P-butyl (h.94), haloxyfop-methyl (h.95), haloxyfop-R-methyl (h.96)), saflufenacil (h.97), sethoxydim (h.98), siduron (h.99), simazine (h.100), simetryn (h.101), sulcotrione (h.102), sulfentrazone (h.103), tebuthiuron (h.104), tembotrione (h.105), tepraloxydim (h.106), terbacil (h.107), terbumeton (h.108), terbuthylazine (h.109), thaxtomin (e.g., the thaxtomins as described in U.S. Pat. No. 7,989,393) (h.110), thenylchlor (h.111), tralkoxydim (h.112), triclopyr (h.113), trietazine (h.114), tropramezone (h.115), and salts and esters thereof; racemic mixtures and resolved isomers thereof, and combinations thereof.

Commercial products containing herbicides are readily available. Herbicide concentration in the composition will generally correspond to the labeled use rate for a particular herbicide.

Non-limiting examples of chemical insecticides, acaricides, and nematicides (“cian”) that may be useful in the seed treatment components used in the methods disclosed herein include carbamates (cian.A), diamides (cian.B), macrocyclic lactones (cian.C), neonicotinoids (cian.D), organophosphates (cian.E), phenylpyrazoles (cian.F), pyrethrins (cian.G), spinosyns (cian.H), synthetic pyrethroids (cian.I), tetronic acids (cian.J) and tetramic acids (cian.K).

In particular embodiments useful chemical insecticides, acaricides, and nematicides include acrinathrin (cian.1), alpha-cypermethrin (cian.2), betacyfluthrin (cian.3), cyhalothrin (cian.4), cypermethrin (cian.5), deltamethrin (cian.6), csfenvalcrate (cian.7), etofenprox (cian.8), fenpropathrin (cian.9), fenvalerate (cian.10), flucythrinat (cian.11), fosthiazate (cian.12), lambda-cyhalothrin (cian.13), gamma-cyhalothrin (cian.14), permethrin (cian.15), tau-fluvalinate (cian.16), transfluthrin (cian.17), zeta-cypermethrin (cian.18), cyfluthrin (cian.19), bifenthrin (cian.20), tefluthrin (cian.21), eflusilanat (cian.22), fubfenprox (cian.23), pyrethrin (cian.24), resmethrin (cian.25), imidacloprid (cian.26), acetamiprid (cian.27), thiamethoxam (cian.28), nitenpyram (cian.29), thiacloprid (cian.30), dinotefuran (cian.31), clothianidin (cian.32), imidaclothiz (cian.33), chlorfluazuron (cian.34), diflubenzuron (cian.35), lufenuron (cian.36), teflubenzuron (cian.37), triflumuron (cian.38), novaluron (cian.39), flufenoxuron (cian.40), hexaflumuron (cian.41), bistrifluoron (cian.42), noviflumuron (cian.43), buprofezin (cian.44), cyromazine (cian.45), methoxyfenozide (cian.46), tebufenozide (cian.47), halofenozide (cian.48), chromafenozide (cian.49), endosulfan (cian.50), fipronil (cian.51), ethiprole (cian.52), pyrafluprole (cian.53), pyriprole (cian.54), flubendiamide (cian.55), chlorantraniliprole (cian.56) (e.g., Rynaxypyr (cian.56a)), cyazypyr (cian.57), emamectin (cian.58), emamectin benzoate (cian.59), abamectin (cian.60), ivermectin (cian.61), milbemectin (cian.62), lepimectin (cian.63), tebufenpyrad (cian.64), fenpyroximate (cian.65), pyridaben (cian.66), fenazaquin (cian.67), pyrimidifen (cian.68), tolfenpyrad (cian.69), dicofol (cian.70), cyenopyrafen (cian.71), cyflumetofen (cian.72), acequinocyl (cian.73), fluacrypyrin (cian.74), bifenazate (cian.75), diafenthiuron (cian.76), etoxazole (cian.77), clofentezine (cian.78), spinosad (cian.79), triarathen (cian.80), tetradifon (cian.81), propargite (cian.82), hexythiazox (cian.83), bromopropylate (cian.84), chinomethionat (cian.85), amitraz (cian.86), pyrifluquinazon (cian.87), pymetrozine (cian.88), flonicamid (cian.89), pyriproxyfen (cian.90), diofenolan (cian.91), chlorfenapyr (cian.92), metaflumizone (cian.93), indoxacarb (cian.94), chlorpyrifos (cian.95), spirodiclofen (cian.96), spiromesifen (cian.97), spirotetramat (cian.98), pyridalyl (cian.99), spinctoram (cian.100), acephate (cian.101), triazophos (cian.102), profenofos (cian.103), oxamyl (cian.104), spinetoram (cian.105), fenamiphos (cian.106), fenamipclothiahos (cian.107), 4-{[6-chloropyrid-3-yl)methyl](2,2-difluoroethyl)amino}furan-2(5H)-one (cian.108), cadusaphos (cian.109), carbaryl (cian.110), carbofuran (cian.111), ethoprophos (cian.112), thiodicarb (cian.113), aldicarb (cian.114), aldoxycarb (cian.115), metamidophos (cian.116), methiocarb (cian.117), sulfoxaflor (cian.118), cyantraniliprole (cian.119), tioxazofen (cian.120), and combinations thereof.

The seed treatment may include in some embodiments commercial seed treatment formulations such as those in Acceleron®. These ingredients may be added as a separate layer on the seed or alternatively may be added as part of the seed coating composition. In one embodiment, where microbial suspensions are applied to corn seeds, the seed treatment components include ipconazole, metalaxyl, trifloxystrobin, and clothianidin. In one embodiment, where microbial suspensions are applied to soy seeds, the seed treatment components include pyraclostrobin, metalaxyl, fluxapyroxad, imidacloprid, fluopyram, clothianidin, Bacillus firmus. In one embodiment, where microbial suspensions are applied to cotton seeds, the seed treatment components include metalaxyl, mycobutanil, trifloxystrobin, pyraclostrobin, imidacloprid, ipconazole, thiamethoxam, abamectin, thiodicarb, ipconazole, and fluxapyroxad.

In some embodiments, the seed treatment pesticides and microorganism compositions can be formulated into a seed treatment. A variety of additives can be added to the seed treatment formulations comprising the isolated, selected, and identified microorganisms. Binders can be added and include those composed of an adhesive polymer that can be natural or synthetic without phytotoxic effect on the seed to be coated. The binder may be selected from polyvinyl acetates; polyvinyl acetate copolymers; ethylene vinyl acetate (EVA) copolymers; polyvinyl alcohols; polyvinyl alcohol copolymers; celluloses, including ethylcelluloses, methylcelluloses, hydroxymethylcelluloses, hydroxypropylcelluloses, and carboxymethylcellulose; polyvinylpyrolidones; polysaccharides, including starch, modified starch, dextrins, maltodextrins, alginate and chitosans; fats; oils; proteins, including gelatin and zeins; gum arabics; shellacs; vinylidene chloride and vinylidene chloride copolymers; calcium lignosulfonates; acrylic copolymers; polyvinylacrylates; polyethylene oxide; acrylamide polymers and copolymers; polyhydroxyethyl acrylate, methylacrylamide monomers; and polychloroprene.

Any of a variety of colorants may be employed, including organic chromophores classified as nitroso; nitro; azo, including monoazo, bisazo and polyazo; acridine, anthraquinone, azine, diphenylmethane, indamine, indophenol, methine, oxazine, phthalocyanine, thiazine, thiazole, triarylmethane, xanthene. Other additives that can be added include trace nutrients such as salts of iron, manganese, boron, copper, cobalt, molybdenum and zinc. A polymer or other dust control agent can be applied to retain the treatment on the seed surface.

In specific embodiments, in addition to the microbial cells or spores, the coating can further comprise a layer of adherent. The adherent should be non-toxic, biodegradable, and adhesive. Examples of such materials include, but are not limited to, polyvinyl acetates; polyvinyl acetate copolymers; polyvinyl alcohols; polyvinyl alcohol copolymers; celluloses, such as methyl celluloses, hydroxymethyl celluloses, and hydroxymethyl propyl celluloses; dextrins; alginates; sugars; molasses; polyvinyl pyrrolidones; polysaccharides; proteins; fats; oils; gum arabics; gelatins; syrups; and starches.

Various additives, such as adherents, dispersants, surfactants, and nutrient and buffer ingredients, can also be included in the seed treatment formulation. Other conventional seed treatment additives include, but are not limited to, coating agents, wetting agents, buffering agents, and polysaccharides. At least one agriculturally acceptable carrier can be added to the seed treatment formulation such as water, solids or dry powders. The dry powders can be derived from a variety of materials such as calcium carbonate, gypsum, fluency agent, vermiculite, talc, humus, activated charcoal, and various phosphorous compounds.

In some embodiments, the seed coating composition can comprise at least one filler which is an organic or inorganic, natural or synthetic component with which the active components are combined to facilitate its application onto the seed. Advantageously, the filler is an inert solid such as clays, natural or synthetic silicates, silica, resins, waxes, solid fertilizers (for example ammonium salts), natural soil minerals, such as kaolins, clays, talc, lime, quartz, attapulgite, montmorillonite, bentonite or diatomaceous earths, or synthetic minerals, such as silica, alumina or silicates, in particular aluminum or magnesium silicates.

### Seed Coating Compositions and Processes

It is contemplated that the seeds can be substantially uniformly coated with one or more layers of the seed treatment and test microbial compositions obtained from the method herein using conventional methods of mixing, spraying or a combination thereof through the use of treatment application equipment that is specifically designed and manufactured to accurately, safely, and efficiently apply seed treatment products to seeds. Such equipment uses various types of coating technology such as rotary coaters, drum coaters, fluidized bed techniques, spouted beds, rotary mists or a combination thereof. Liquid seed treatments such as those of the methods described herein can be applied via either a spinning “atomizer” disk or a spray nozzle which evenly distributes the seed treatment onto the seed as it moves though the spray pattern. Advantageously, the seed is then mixed or tumbled for an additional period of time to achieve additional treatment distribution and drying. The seeds can be primed or unprimed before coating with the inventive compositions to increase the uniformity of germination and emergence. In an alternative embodiment, a dry powder formulation can be metered onto the moving seed and allowed to mix until completely distributed.

Generally, the amount of the isolated, selected, and identified microorganisms or other ingredients used in the seed treatment, should not inhibit germination of the seed, or cause phytotoxic damage to the seed.

The microorganism and seed treatment treated seeds may also be further enveloped with a film overcoating to protect the coating. Such overcoatings are known in the art and may be applied using fluidized bed and drum film coating techniques.

### Plants Suitable for the Methods of the Invention

In principle, the methods according to the present invention can be deployed to identify seed treatment tolerant microorganisms associated with and useful in seed treatments for seeds of any plant species. Monocotyledonous as well as dicotyledonous plant species are particularly suitable. The methods and compositions are often used with plants that are important or interesting for agriculture, horticulture, for the production of biomass used in producing liquid fuel molecules and other chemicals, and/or forestry.

Thus, the methods described herein have use over a broad range of plants, including higher plants pertaining to the classes of Angiospermae and Gymnospermae. Plants of the subclasses of the Dicotylodenae and the Monocotyledonae are particularly suitable. Dicotyledonous plants belong to the orders of the Aristochiales, Asterales, Batales, Campanulales, Capparales, Caryophyllales, Casuarinales, Celastrales, Cornales, Diapensales, Dilleniales, Dipsacales, Ebenales, Ericales, Eucomiales, Euphorbiales, Fabales, Fagales, Gentianales, Geraniales, Haloragales, Hamamelidales, Illiciales, Juglandales, Lamiales, Laurales, Lecythidales, Leitneriales, Magniolales, Malvales, Myricales, Myrtales, Nymphaeales, Papeverales, Piperales, Plantaginales, Plumbaginales, Podostemales, Polemoniales, Polygalales, Polygonales, Primulales, Proteales, Rafflesiales, Ranunculales, Rhamnales, Rosales, Rubiales, Salicales, Santales, Sapindales, Sarraceniaceae, Scrophulariales, Theales, Trochodendrales, Umbellales, Urticales, and Violales. Monocotyledonous plants belong to the orders of the Alismatales, Arales, Arecales, Bromeliales, Commelinales, Cyclanthales, Cyperales, Eriocaulales, Hydrocharitales, Juncales, Lilliales, Najadales, Orchidales, Pandanales, Poales, Restionales, Triuridales, Typhales, and Zingiberales. Plants belonging to the class of the Gymnospermae are Cycadales, Ginkgoales, Gnetales, and Pinales.

Suitable species may include members of the genus Abelmoschus, Abies, Acer, Agrostis, Allium, Alstroemeria, Ananas, Andrographis, Andropogon, Artemisia, Arundo, Atropa, Berberis, Beta, Bixa, Brassica, Calendula, Camellia, Camptotheca, Cannabis, Capsicum, Carthamus, Catharanthus, Cephalotaxus, Chrysanthemum, Cinchona, Citrullus, Coffea, Colchicum, Coleus, Cucumis, Cucurbita, Cynodon, Datura, Dianthus, Digitalis, Dioscorea, Elaeis, Ephedra, Erianthus, Erythroxylum, Eucalyptus, Festuca, Fragaria, Galanthus, Glycine, Gossypium, Helianthus, Hevea, Hordeum, Hyoscyamus, Jatropha, Lactuca, Linum, Lolium, Lupinus, Lycopersicon, Lycopodium, Manihot, Medicago, Mentha, Miscanthus, Musa, Nicotiana, Oryza, Panicum, Papaver, Parthenium, Pennisetum, Petunia, Phalaris, Phleum, Pinus, Poa, Poinsettia, Populus, Rauwolfia, Ricinus, Rosa, Saccharum, Salix, Sanguinaria, Scopolia, Secale, Solanum, Sorghum, Spartina, Spinacea, Tanacetum, Taxus, Theobroma, Triticosecale, Triticum, Uniola, Veratrum, Vinca, Vitis, and Zea.

The methods described herein may be used for identification of seed treatment toleranct microorganisms on seeds from plants that are important or interesting for agriculture, horticulture, biomass for the production of biofuel molecules and other chemicals, and/or forestry. Non-limiting examples include, for instance, Panicum virgatum (switchgrass), Sorghum bicolor (sorghum, sudangrass), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), Pennisetum glaucum (pearl millet), Panicum spp., Sorghum spp., Miscanthus spp., Saccharum spp., Erianthus spp., Populus spp., Andropogon gerardii (big bluestem), Pennisetum purpureum (elephant grass), Phalaris arundinacea (reed canarygrass), Cynodon dactylon (bermudagrass), Festuca arundinacea (tall fescue), Spartina pectinata (prairie cord-grass), Arundo donax (giant reed), Secale cereale (rye), Salix spp. (willow), Eucalyptus spp. (eucalyptus), Triticosecale spp. (triticum-wheat X rye), Bamboo, Carthamus tinctorius (safflower), Jatropha curcas (Jatropha), Ricinus communis (castor), Elaeis guineensis (oil palm), Phoenix dactylifera (date palm), Archontophoenix cunninghamiana (king palm), Syagrus romanzoffiana (queen palm), Linum usitatissimum (flax), Brassica juncea, Manihot esculenta (cassaya), Lycopersicon esculentum (tomato), Lactuca saliva (lettuce), Musa paradisiaca (banana), Solanum tuberosum (potato), Brassica oleracea (broccoli, cauliflower, brusselsprouts), Camellia sinensis (tea), Fragaria ananassa (strawberry), Theobroma cacao (cocoa), Coffea arabica (coffee), Vitis vinifera (grape), Ananas comosus (pineapple), Capsicum annum (hot & sweet pepper), Allium cepa (onion), Cucumis melo (melon), Cucumis sativus (cucumber), Cucurbita maxima (squash), Cucurbita moschata (squash), Spinacea oleracea (spinach), Citrullus lanatus (watermelon), Abelmoschus esculentus (okra), Solanum melongena (eggplant), Papaver somniferum (opium poppy), Papaver orientale, Taxus baccata, Taxus brevifolia, Artemisia annua, Cannabis saliva, Camptotheca acuminate, Catharanthus roseus, Vinca rosea, Cinchona officinalis, Coichicum autumnale, Veratrum californica, Digitalis lanata, Digitalis purpurea, Dioscorea spp., Andrographis paniculata, Atropa belladonna, Datura stomonium, Berberis spp., Cephalotaxus spp., Ephedra sinica, Ephedra spp., Erythroxylum coca, Galanthus wornorii, Scopolia spp., Lycopodium serratum (Huperzia serrata), Lycopodium spp., Rauwolfia serpentina, Rauwolfia spp., Sanguinaria canadensis, Hyoscyamus spp., Calendula officinalis, Chrysanthemum parthenium, Coleus forskohlii, Tanacetum parthenium, Parthenium argentatum (guayule), Hevea spp. (rubber), Mentha spicata (mint), Mentha piperita (mint), Bixa orellana, Alstroemeria spp., Rosa spp. (rose), Dianthus caryophyllus (carnation), Petunia spp. (petunia), Poinsettia pulcherrima (poinsettia), Nicotiana tabacum (tobacco), Lupinus albus (lupin), Uniola paniculata (oats), bentgrass (Agrostis spp.), Populus tremuloides (aspen), Pinus spp. (pine), Abies spp. (fir), Acer spp. (maple), Hordeum vulgare (barley), Poa pratensis (bluegrass), Lolium spp. (ryegrass), Phleum pratense (timothy), and conifers. In other embodiments, plants used in the method herein are grown for energy production, so called energy crops, such as cellulose-based energy crops like Panicum virgatum (switchgrass), Sorghum bicolor (sorghum, sudangrass), Miscanthus giganteus (miscanthus), Saccharum sp. (energycane), Populus balsamifera (poplar), Andropogon gerardii (big bluestem), Pennisetum purpureum (elephant grass), Phalaris arundinacea (reed canarygrass), Cynodon dactylon (bermudagrass), Festuca arundinacea (tall fescue), Spartina pectinata (prairie cord-grass), Medicago sativa (alfalfa), Arundo donax (giant reed), Secale cereale (rye), Salix spp. (willow), Eucalyptus spp. (eucalyptus), Triticosecale spp. (triticum-wheat X rye), and Bamboo; and starch-based energy crops like Zea mays (corn) and Manihot esculenta (cassava); and sugar-based energy crops like Saccharum sp. (sugarcane), Beta vulgaris (sugarbeet), and Sorghum bicolor (L.) Moench (sweet sorghum); and biofuel-producing energy crops like Glycine max (soybean), Brassica napus (canola), Helianthus annuus (sunflower), Carthamus tinctorius (safflower), Jatropha curcas (Jatropha), Ricinus communis (castor), Elaeis guineensis (African oil palm), Elaeis oleifera (American oil palm), Cocos nucifera (coconut), Camelina sativa (wild flax), Pongamia pinnata (Pongam), Olea europaea (olive), Linum usitatissimum (flax), Crambe abyssinica (Abyssinian-kale), and Brassica juncea.

Throughout this disclosure, various information sources are referred to and incorporated by reference. The information sources include, for example, scientific journal articles, patent documents, and textbooks. The reference to such information sources is solely for the purpose of providing an indication of the general state of the art at the time of filing. While the contents and teachings of each and every one of the information sources can be relied on and used by one of skill in the art to make and use embodiments of the invention, any discussion and comment in a specific information source should in no way be considered as an admission that such comment was widely accepted as the general opinion in the field.

All publications and patent applications mentioned in this specification are herein incorporated by reference to the same extent as if each individual publication or patent application was specifically and individually indicated to be incorporated by reference.

No admission is made that any reference constitutes prior art. The discussion of the references states what their authors assert, and the applicants reserve the right to challenge the accuracy and pertinence of the cited documents. It will be clearly understood that, although a number of prior art publications are referred to herein, this reference does not constitute an admission that any of these documents forms part of the common general knowledge in the art.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following examples are merely illustrative, and do not limit this disclosure in any way.

### Example 1

### Example 1A

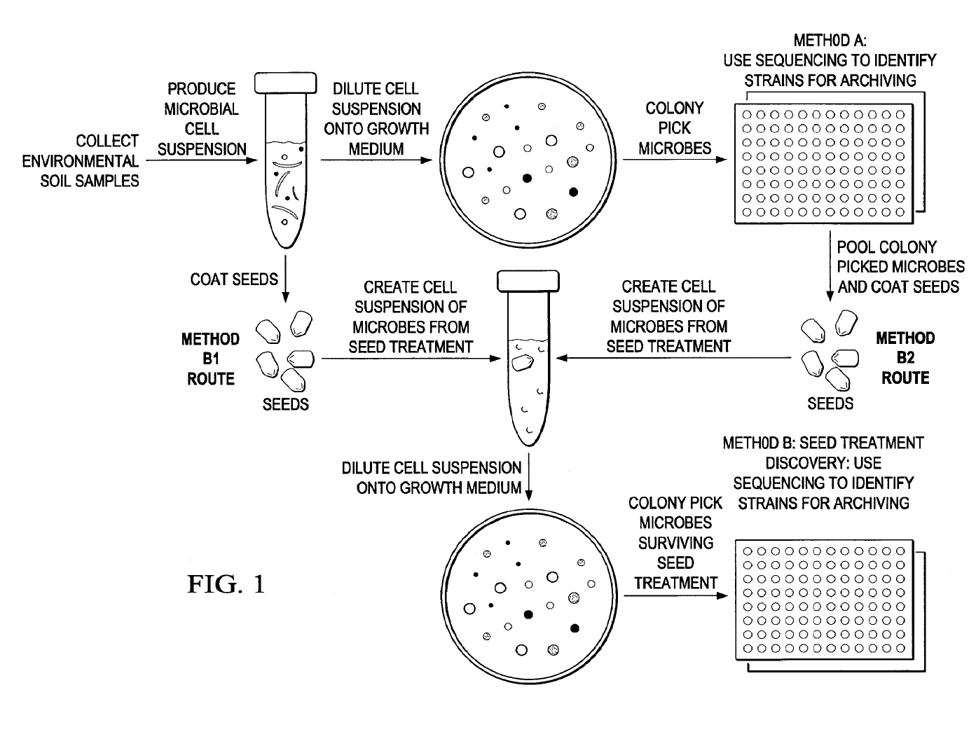

This example used microbial cell suspensions as source material. The cell suspensions were a representative extracts of all of the bacteria, fungi, and archaea present in a microbiota (Method B1 of FIG. 1), including, but not limited to, those found in soils, plant tissues, and bodies of water. These cell suspensions were used to inoculate seeds in order to identify those capable of surviving the seed treatment process.

The cell suspensions were derived from 50 mL of starting microbial cell suspension centrifuged at 500 RCF to pellet debris. The supernatant was decanted from the sample and passed through a filter to further remove debris. The filtered supernatant was then centrifuged at 16K RCF for 20 minutes to produce a pellet consisting primarily of cellular material. The supernatant was discarded and the resulting pellet was re-suspended into 1 mL sterile PBS to concentrate the cellular fraction.

The resulting cell suspension was further concentrated by transferring 1 mL to a 1.5 mL micro centrifuge tube and spinning the sample at 14K RCF for 10 minutes. 850 μL of the supernatant was removed and the pellet was re-suspended into the remaining supernatant. 100 μL of this highly concentrated sample was added to 40 corn seeds (DKC62-61) contained in a sterile 50 mL centrifuge tube.

The seeds and concentrated cell suspension were shaken vigorously and vortexed for 30 seconds. 65 μL of Precise Seed Finisher® 1006, diluted 1/5 in water, was then added to the seed/cell suspension mixture and shaken/vortexed for 30 seconds to ensure uniform coverage. The corn seeds treated with the microbial suspension were transferred to a sterile dish and allowed to dry for 20 minutes inside a biosafety cabinet. The treated and dried seeds were stored at room temperature (−22° C.) overnight and single seeds were then added to 10 mL sterile PBS contained in 50 mL centrifuge tubes, producing a 1/10 dilution of the material contained on the seed. The replicate samples were vortexed for 30 seconds, allowed to sit for one hour, then vortexed again for 30 seconds to ensure removal of all cells adhered to the seeds. The suspension was further diluted 1/10 and 1/100, and 100 μL of each microbial seed treatment replicate and dilution series was spread onto R2A agar growth medium in 150 mm Petri dishes and incubated at room temperature. Microbial colonies appeared on the agar over the course of 1-10 days, and these colonies were picked into 96-well microtiter plates with wells containing 150 μL R2B+YT growth medium.

After reaching turbidity, the individual cultures contained in the 96-well plates were submitted to PCR and taxonomic identification of isolated strains. These microbial strains were considered “seed treatment survivors.” The strains were then de-replicated as follows. Sequences were compared to each other using multiple sequence comparison by log-expectation (MUSCLE) alignments and phylogenic trees to select only those strains for archiving that were unique within the sample. Generally, sequences differing from each other by 0.5% or more across the length of the gene were considered unique. De-replication, the rapid identification of known organisms in a mixture, can be done using a number of methods known to those of skill in the art.

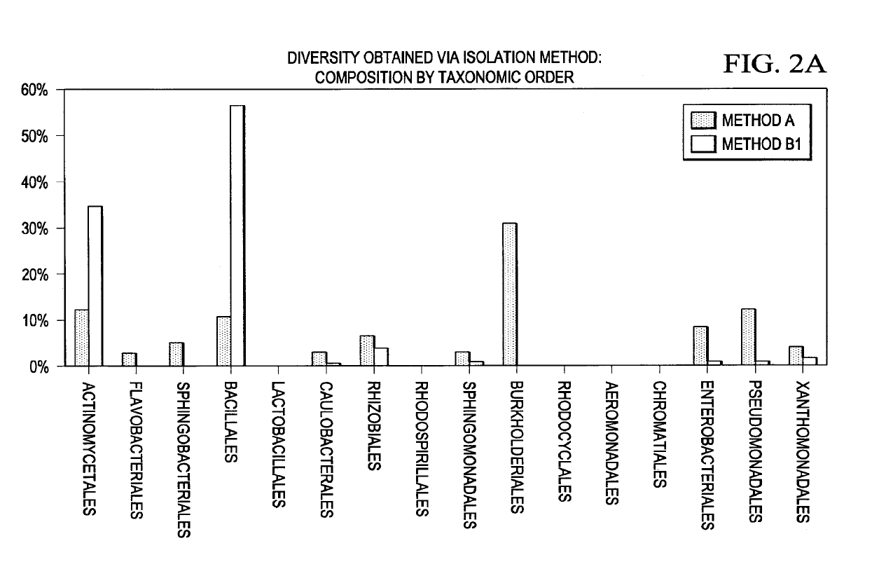

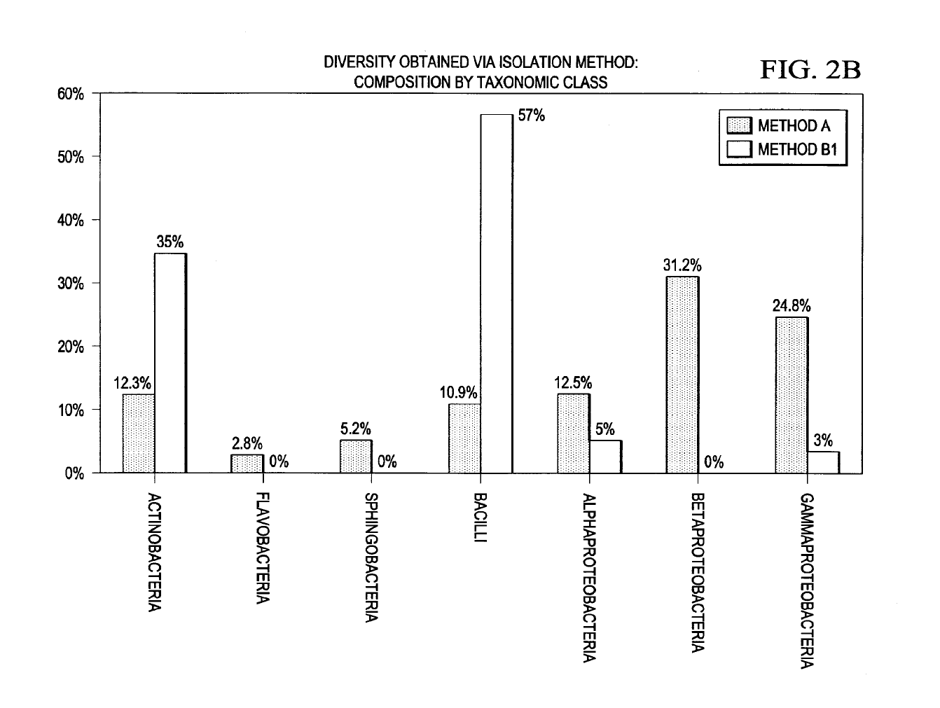

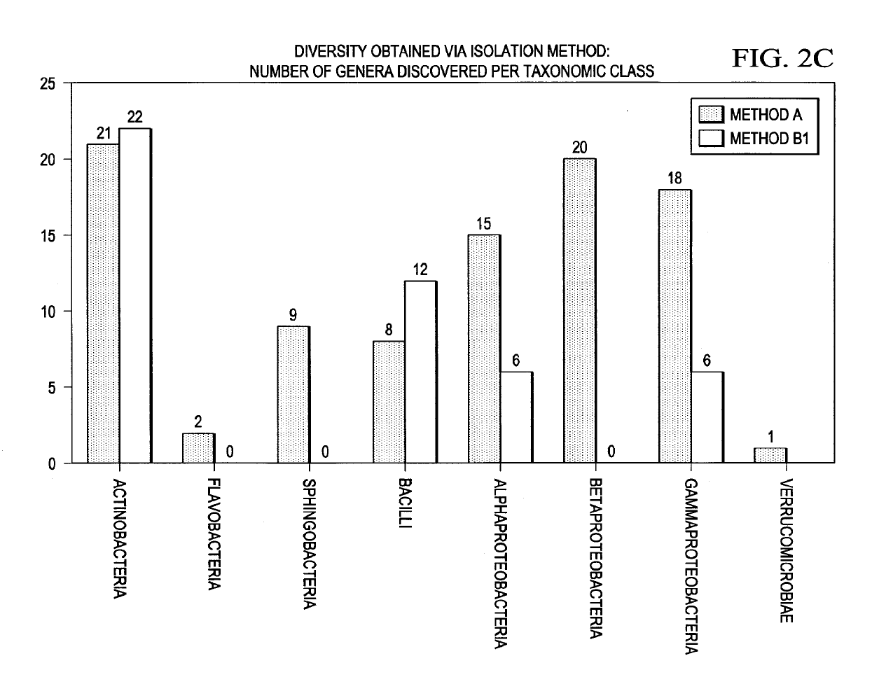

The results of this study are given in FIGS. 2A-C. The data marked as “Method A” in FIGS. 2A-C represent the non-high throughput method involving cell suspensions not used to treat seeds. Data marked as “Method B” represent data generated Method B1. As can be seen, using this approach, multiple spore-forming species of bacteria were identified as viable post seed-treatment, while the method successfully removed “non-survivors,” enriching for seed-treatment tolerant microbes. Unexpectedly, a diverse collection of non-spore forming microbes were also identified.

### Example 1B

This example used microbial cell suspensions as source material. The cell suspension was a cell suspension derived from an artificially assembled pool of microbes that were cultured previously (Method B2 of FIG. 1). These cell suspensions were used to inoculate seeds in order to identify those capable of surviving the seed treatment process.

Existing microbial cultures were pooled together and a cell suspension of microbes was created, as set forth in Method B2. The seeds and concentrated cell suspension were shaken vigorously and vortexed for 30 seconds. 65 μL of Precise Seed Finisher® 1006, diluted 1/5 in water, was then added to the seed/cell suspension mixture and shaken/vortexed for 30 seconds to ensure uniform coverage. The corn seeds treated with the microbial suspension were transferred to a sterile dish and allowed to dry for 20 minutes inside a biosafety cabinet. The treated and dried seeds were stored at room temperature (−22° C.) overnight and single seeds were then added to 10 mL sterile PBS contained in 50 mL centrifuge tubes, producing a 1/10 dilution of the material contained on the seed. The replicate samples were vortexed for 30 seconds, allowed to sit for one hour, then vortexed again for 30 seconds to ensure removal of all cells adhered to the seeds. The suspension was further diluted 1/10 and 1/100, and 100 μL of each microbial seed treatment replicate and dilution series was spread onto R2A agar growth medium in 150 mm Petri dishes and incubated at room temperature. Microbial colonies appeared on the agar over the course of 1-10 days, and these colonies were picked into 96-well microtiter plates with wells containing 150 μL R2B+YT growth medium.

After reaching turbidity, the individual cultures contained in the 96-well plates were submitted to PCR and taxonomic identification of isolated strains. These microbial strains were considered “seed treatment survivors.” The strains were then de-replicated as follows. Sequences were compared to each other using multiple sequence comparison by log-expectation (MUSCLE) alignments and phylogenic trees to select only those strains for archiving that were unique within the sample. Generally, sequences differing from each other by 0.5% or more across the length of the gene were considered unique. De-replication, the rapid identification of known organisms in a mixture, can be done using a number of methods known to those of skill in the art.

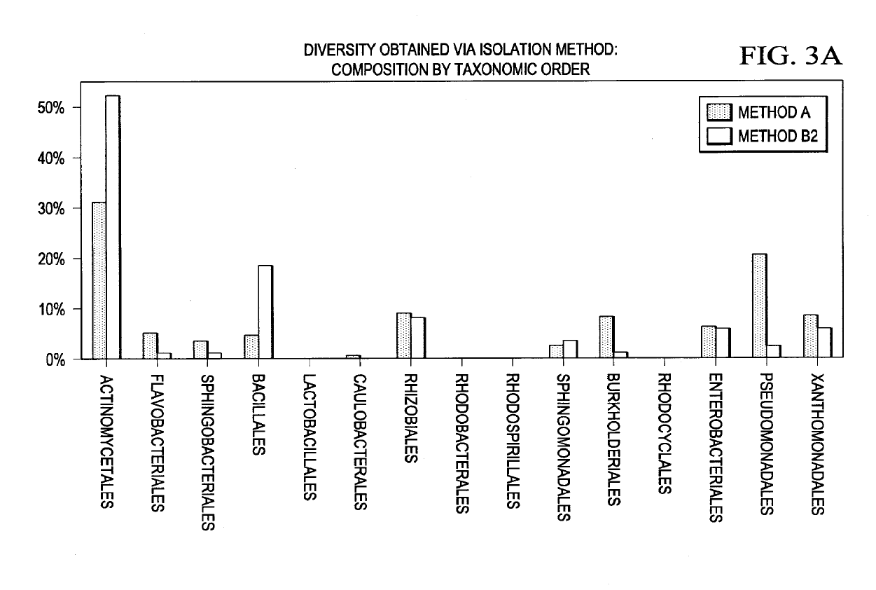

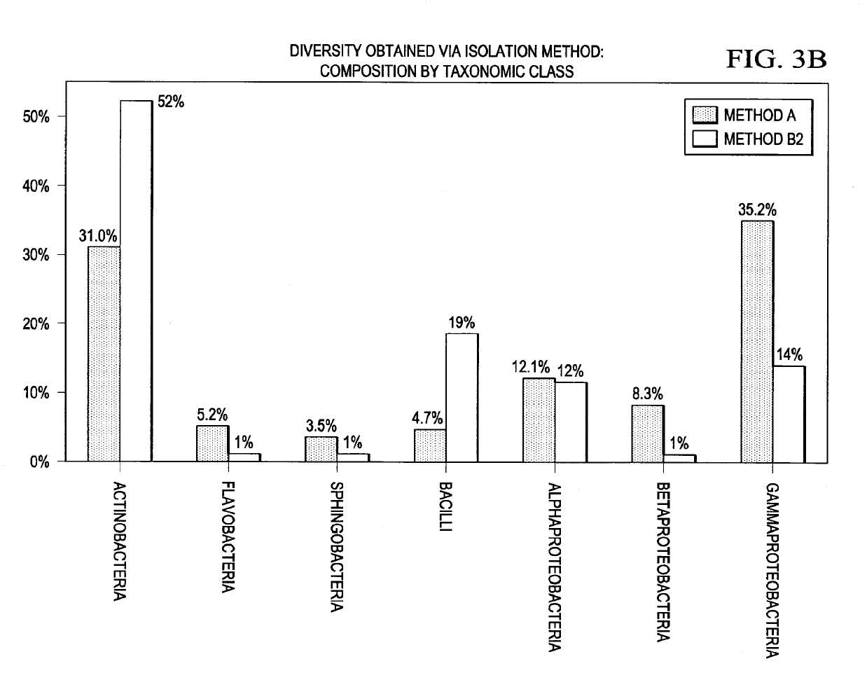

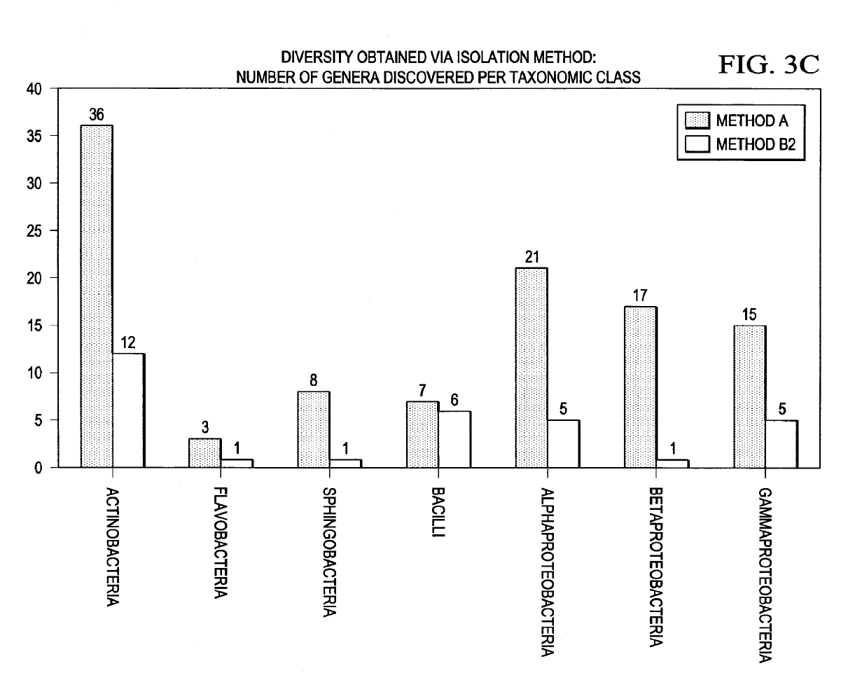

The results of this study are given in FIGS. 3A-3C. The data marked as “Method A” in FIGS. 3A-3C represents the non-high-throughput method involving cell suspensions not used to treat seeds. Data marked as “Method B” represent a composite of the data generated using either Method B2. As can be seen, using this approach, multiple spore-forming species of bacteria were identified as viable post seed-treatment, while the method successfully removed “non-survivors,” enriching for seed-treatment tolerant microbes. Unexpectedly, a diverse collection of non-spore forming microbes were also identified.

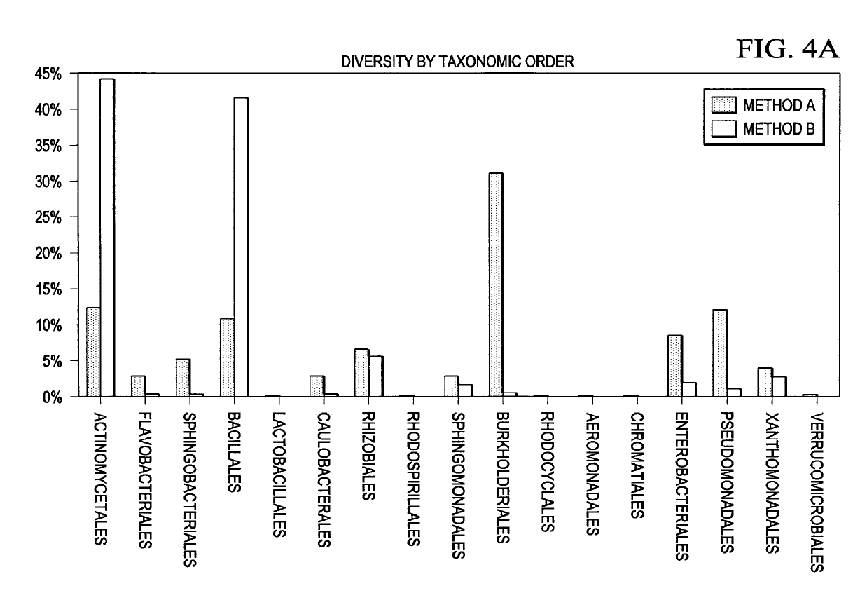

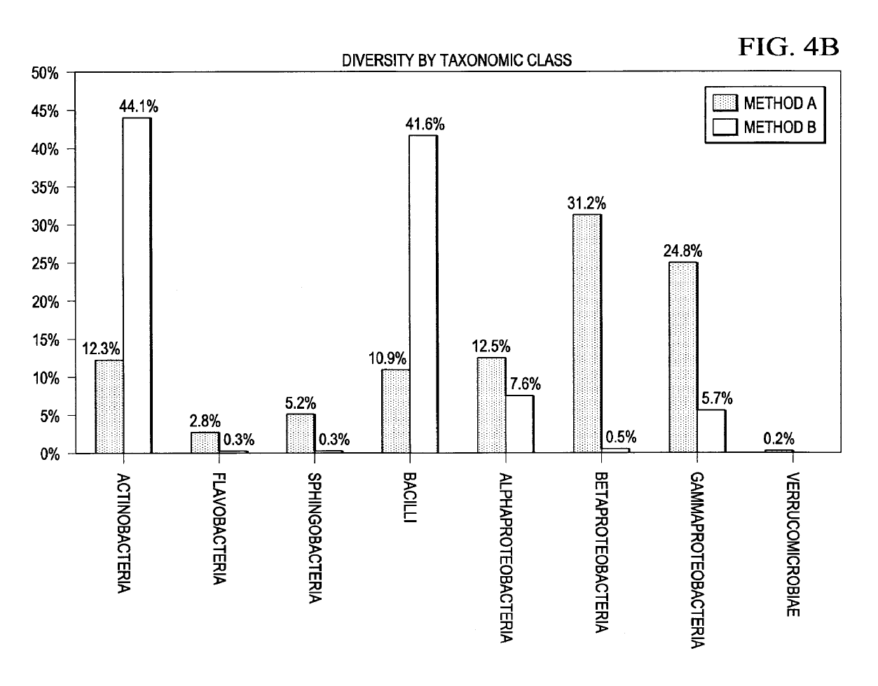

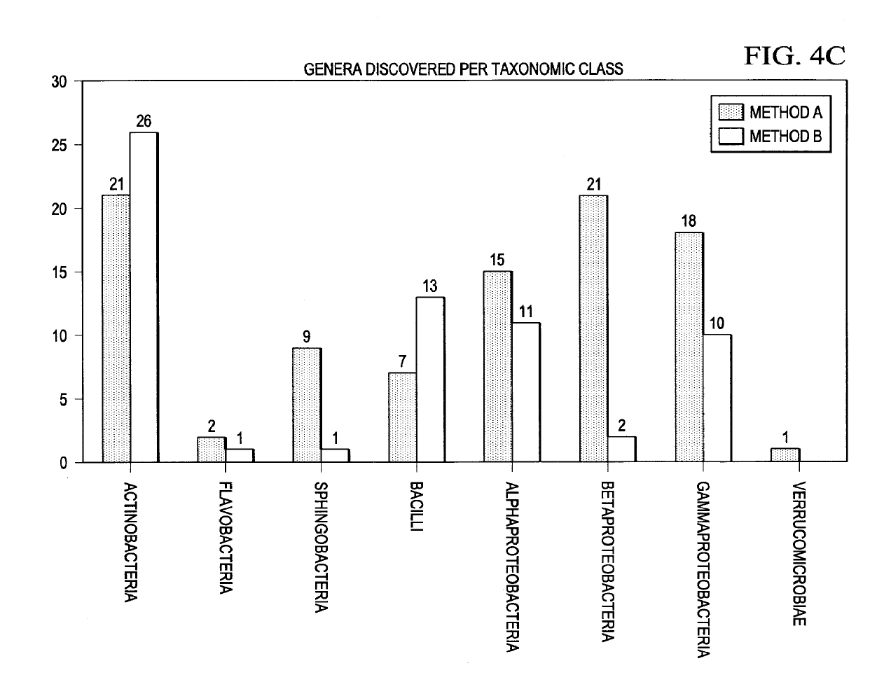

The results of Examples 1A and 1B are combined and given in FIGS. 4A-C. The data marked as “Method A” in FIGS. 2A-C represent non-high-throughput cell suspensions not used to treat seeds. Data marked as “Method B” represent a combination of the data from Examples 1A and 1B. As can be seen, using this approach, multiple spore-forming species of bacteria were identified as viable post seed-treatment, while the method successfully removed “non-survivors,” enriching for seed-treatment tolerant microbes. Unexpectedly, a diverse collection of non-spore forming microbes were also identified.

### Example 2

This example uses as a microbial cell suspension its source material. The cell suspension can be a representative extract of all of the bacteria, fungi, and archaea present in a microbiota, including, but not limited to, those found in soils, plant tissues, and bodies of water (Method B1), or a cell suspension can also be derived from an artificially assembled pool of microbes that are cultured previously (Method B2).

50 mL or more of the sample is centrifuged at 500 RCF to pellet debris. The supernatant is decanted from the sample and passed through a filter to further remove debris. The filtered supernatant is then centrifuged at 16K RCF for 20 minutes to produce a pellet consisting primarily of cellular material. The supernatant is discarded and the resulting pellet is re-suspended into 1 mL sterile PBS to concentrate the cellular fraction. Artificially assembled cell suspensions of cultured microorganisms may not require this concentration and filtration step.

The resulting cell suspension is further concentrated by transferring 1 mL to a 1.5 mL micro centrifuge tube and spinning the sample at 14K RCF for 10 minutes. 850 μL of the supernatant is removed and the pellet is re-suspended into the remaining supernatant. 100 μL of this highly concentrated sample is added to 40 corn seeds treated with chemical seed treatment (hereinafter “seeds”) contained in a sterile 50 mL centrifuge tube. The seeds and concentrated cell suspension are shaken vigorously and vortexed for 30 seconds. 65 μL of Precise Seed Finisher® 1006, diluted 1/5 in water, is then added to the seed/cell suspension mixture and shaken/vortexed for 30 seconds to ensure uniform coverage. The corn seeds treated with the microbial suspension are transferred to a sterile dish and allowed to dry for 20 minutes inside a biosafety cabinet.

The treated and dried seeds are stored at room temperature (−22° C.) overnight and single seeds are then added to 10 mL sterile PBS contained in 50 mL centrifuge tubes, producing a 1/10 dilution of the material contained on the seed. The replicate samples are vortexed for 30 seconds, allowed to sit for one hour, then vortexed again for 30 seconds to ensure removal of all cells adhered to the seeds. The suspension is further diluted 1/10 and 1/100, and 100 μL of each microbial seed treatment replicate and dilution series is spread onto R2A agar growth medium in 150 mm Petri dishes and incubated at room temperature. Microbial colonies appeare on the agar over the course of 1-10 days, and these colonies are picked into 96-well microtiter plates with wells containing 150 μL R2B+YT growth medium.

After reaching turbidity, the individual cultures contained in the 96-well plates are submitted to PCR and taxonomic identification of isolated strains. These microbial strains are considered “seed treatment survivors.” The strains are then de-replicated as follows. Sequences are compared to each other using multiple sequence comparison by log-expectation (MUSCLE) alignments and phylogenic trees to select only those strains for archiving that are unique to the sample. Generally, sequences differing from each other by 0.5% or more across the length of the gene are considered unique. De-replication, the rapid identification of known organisms in a mixture, can be done using a number of methods known to those of skill in the art.

### Example 3

Purification and concentration of the isolated microbial cell suspension, as described supra, can be improved to recover less-abundant microbes. For example, sonicated root extracts are dilute and contain a large quantity of plant/soil debris. As a result, the number of initial CFUs incorporated into the seed treatment for isolation is low and this can affect how many microorganisms can be recovered post-seed treatment. However, creating a pooled microbial cell suspension from multiple replicates of larger plants can increase the number of environmental microbes isolated. This larger microbial cell suspension can then be purified using density gradient centrifugation to improve upon the post-seed treatment recovery rate of those microorganisms less abundant in the environmentally-isolated, wild-type microbial populations.

1. A high-throughput method for obtaining a microorganism comprising the steps of:
a) obtaining a plurality of microorganisms;
b) applying the plurality of microorganisms to a seed or surrogate thereof;
c) storing said seed, or surrogate thereof under conditions wherein one or more members of said plurality of microorganisms becomes inviable;
d) placing said seed, or surrogate thereof, in a solution; and
e) identifying from said solution at least a first microorganism remaining viable following said step (c).

2. The method of claim 1, wherein said step (a) comprises associating said plurality of microorganisms from a growth environment with a crop plant.

3. The method of claim 1, wherein said step (a) comprises generating a microbial cell suspension from said plurality of microorganisms.

4. The method of claim 3, comprising concentrating said microbial cell suspension prior to said step (b).

5. The method of claim 1, wherein said step (e) comprises plating said solution onto a growth medium and selecting a colony comprising the microorganism.

6. The method of claim 1, wherein said step (a) comprises:
i) generating a microbial cell suspension;
ii) plating the microbial cell suspension onto a growth medium;
iii) selecting microbial colonies; and
iv) producing the plurality of microorganisms by combining members of the selected microbial colonies in step iii).

7. The method of claim 2, wherein said growth environment comprises soil from an agricultural field.

8. The method of claim 2, wherein said growth environment is a non-agriculture environment.

9. The method of claim 1, further comprising the step of:
f) identifying at least a first beneficial trait the microorganism is capable of conferring upon plants of a crop plant species.

10. The method of claim 9, wherein the seed is of the same species as the crop plant species.

11. The method of claim 1, wherein said plurality of microorganisms are from a crop plant rhizosphere, endosphere, phyllosphere, or any combination thereof.

12. The method of claim 3, wherein said microbial cell suspension is obtained from tissue of said crop plant.

13. The method of claim 1, wherein the crop plant is a dicotyledonous plant.

14. The method of claim 13, wherein the dicotyledonous plant is selected from the group consisting of alfalfa, beans, beet, broccoli, cabbage, carrot, cauliflower, celery, Chinese cabbage, cotton, cucumber, eggplant, flax, lettuce, lupine, melon, pea, pepper, peanut, potato, pumpkin, radish, rapeseed, spinach, soybean, squash, sugarbeet, sunflower, tomato, and watermelon.

15. The method of claim 1, wherein the crop plant is a monocotyledonous plant.

16. The method of claim 15, wherein the monocotyledonous plant is selected from the group consisting of barley, corn, leek, onion, rice, sorghum, sweet corn, wheat, rye, millet, sugarcane, oat, triticale, switchgrass, and turfgrass.

17. The method of claim 1, wherein the microorganism remaining viable is a gram-negative, non-spore forming bacterium.

18. The method of claim 1, wherein the microorganism remaining viable is a gram-positive, spore forming or non-spore forming bacterium.

19. The method of claim 1, wherein storing said seed, or surrogate thereof, is carried out for from about 1 hour to about 1 year.

20. The method of claim 1, wherein storing said seed, or surrogate thereof, is carried out at ambient temperature.

21. The method of claim 1, wherein storing said seed, or surrogate thereof, is carried out at above or below ambient temperature.

22. The method of claim 1, wherein step (b) further comprises applying a seed treatment to said seed or surrogate thereof prior to, concurrently with, or after applying said plurality of microorganisms.

23. The method of claim 22, wherein the seed treatment comprises the plurality of beneficial microorganisms.

24. The method of claim 22, wherein the seed treatment comprises a chemical pesticide.

25. The method of claim 22, wherein said seed treatment is applied to the seed or surrogate thereof prior to or after applying said plurality of microorganisms.

26. The method of claim 22, wherein the seed treatment comprises a fungicide, biocide, insecticide, herbicide, miticide, rodenticide, nematicides, plant growth regulators, and micronutrients, or a combination thereof.

27. The method of claim 22, wherein the seed treatment comprises a polymer, colorant, binder, adhesive, adherent, dispersant, surfactant, nutrient, coating agent, wetting agent, buffering agent, polysaccharide, and filler, or a combination thereof.
